(19) United States
(12) Patent Application Publication
(10) Pub. No.: US 2024/0084257 A1
(43) Pub. Date: Mar. 14, 2024

(54) REAGENTS AND METHODS FOR PRODUCING ERYTHROMYELOID PROGENITOR CELLS, NK CELLS, AND MEGAKARYOCYTES

(71) Applicant: Wisconsin Alumni Research Foundation, Madison, WI (US)

(72) Inventors: Igor Slukvin, Madison, WI (US); Ho Sun Jung, Madison, WI (US)

(21) Appl. No.: 18/463,211

(22) Filed: Sep. 7, 2023

Related U.S. Application Data
(60) Provisional application No. 63/404,508, filed on Sep. 7, 2022.

(51) Int. Cl. C12N 5/0783; A61K 35/17; A61K 35/19; C12N 5/078

(52) U.S. Cl. CPC C12N 5/0646; A61K 35/17; A61K 35/19; C12N 5/0644; C12N 2501/60; C12N 2506/45

(57) ABSTRACT

This disclosure provides reagents and methods for producing NK progenitor cells, NK cells, megakaryocytes and CAR derivatives thereof from hematopoietic stem cells. Pharmaceutical compositions comprising NK progenitor cells produced by the reagents and methods of the invention and immunotherapeutic methods using these pharmaceutical compositions are also provided.

### FEDERALLY SPONSORED RESEARCH

This invention was made with government support under HL142665, OD011106 and HL134655 awarded by the National Institutes of Health. The government has certain rights in the invention.

### STATEMENT REGARDING SEQUENCE LISTING

A computer readable form of the Sequence Listing is filed with this application by electronic submission and is incorporated into this application by reference in its entirety. The Sequence Listing is contained in the file created on Sep. 5, 2023, having the file name “22-0629-US.xml” and is 37 kb in size.

### BACKGROUND OF THE DISCLOSURE

Elucidation of developmental pathways using human embryonic stem cells and induced pluripotent stem cells over the past 25 years have enabled production of therapeutically useful amounts of specific cell types that can, inter alia, provide allogenic sources of cells specific for individual patients. A specific example of such cell types is produced during hematopoiesis to provide a number of important components of blood function and homeostasis, including for example cells involved in immunological surveillance and function. One such cell is the natural killer or NK cell, a species of white blood cell in the lymphoid lineage which is characterized by having granules containing enzymes that can kill tumor cells or cells infected with a virus. Yet another are megakaryocytes, cells found in bone marrow that are responsible for producing thrombocytes (platelets).

Another consequence of the ability to study and manipulate stem cells and particularly hematopoietic stem cells has been an understanding of endogenous proteins produced during differentiation that effect progression to final effector cells like NK cells. These proteins, including transcription factors are responsible for activating specific genes in developmental pathways leading to particular differentiation outcomes. One example of such transcription factors is the SOXF family of transcription factors, comprising SOX7, SOX17 and SOX18, that have been recognized as critical regulators of angiogenesis, cardiovascular and hematopoietic development. See, for example, Lilly et al., 2017 SOXF transcription factors in cardiovascular development. Seminars in cell & developmental biology 63: 50-57.

Murine embryonic studies have shown that Sox7 is required for formation of the earliest multipotent hematopoietic progenitor cells (HPs) with erythro-myeloid potential. Gandillet et al., 2009, Sox7-sustained expression alters the balance between proliferation and differentiation of hematopoietic progenitors at the onset of blood specification. Blood.114: 4813-4822. Forced expression of Sox7 in cells from E7.5 mouse embryo or from in vitro differentiated mouse embryonic stem cells (ESCs) promotes self-renewal of early CD41⁺ hemogenic progenitors with erythro-myeloid potential and blocks their differentiation. Lilly et al., 2016, Interplay between SOX7 and RUNX1 regulates hemogenic endothelial fate in the yolk sac. Development 143: 4341-4351. A similar phenotype was observed following overexpression of Sox18 in in vitro-differentiated mouse ESCs. Serrano et al., 2010, Contrasting effects of Sox17- and Sox 1 8-sustained expression at the onset of blood specification. Blood 115: 3895-3898. SOX17 has been shown using human embryonic stem cells to be a master regulator of HOXA and arterial programs in hemogenic endothelium (HE) and is required for the specification of HE with robust lympho-myeloid potential and DLL4⁺CXCR4⁺ phenotype resembling arterial HE at sites of HSC emergence. Jung et al., 2021, SOX17 integrates HOXA and arterial programs in hemogenic endothelium to drive definitive lympho-myeloid hematopoiesis. Cell Rep. 34(7):108758.

There is a need in this art to further elucidate functions of transcription factors in the SOXF family to develop reagents and methods for manipulating hematopoiesis in human stem cells to produce cells for therapeutic intervention and other purposes.

### SUMMARY OF THE DISCLOSURE

Provided herein are reagents and methods for manipulating pluripotent stem cells, and particularly human stem cells, for producing specific cell types resulting from hematopoietic differentiation. In specific embodiments, such cells are natural killer (NK) cells. In other specific embodiments are megakaryocytes. Specific embodiments of the methods provided herein result in enforced SOX18 expression in stem cells and hemogenic endothelial cells during the endothelial-to-hematopoietic transition. In particular, such enforced SOX18 expression produces preferential commitment to NK progenitor cells. In specific embodiments such NK progenitor cells are derived from multipotent hematopoietic progenitors having a phenotype of CD34⁺CD43⁺CD235a/CD41a⁻CD45⁻. In particular embodiments the invention provides such cells, inter alia, for use in immunotherapy.

Yet another aspect of the disclosure provides a pharmaceutical composition comprising NK cells produced by forced expression of SOX18 in hemogenic endothelium. In some embodiments the NK cell is a genetically modified cell such as a CAR-NK cell. In certain embodiments, CAR-encoding expression constructs are introduced into pluripotent stem cells (PSCs) and those cells differentiated to CAR-NK cells by forced expression of SOX18. In alternative embodiments NK cells are produced from PSCs and CAR-encoding expression constructs introduced into the NK cells thereby produced.

In still further aspects of the disclosure are provided megakaryocytes through forced expression of SOX18 in mesoderm and hemogenic endothelium.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### DESCRIPTION OF THE DRAWINGS

The disclosure will be better understood and features, aspects, and advantages other than those set forth above will become apparent when consideration is given to the following detailed description thereof. Such detailed description refers to the following drawings.

FIG. 1 is a diagram of vectors used in the PiggyBac system to generate DOX-inducible SOX18 in an H1 hESC line.

FIGS. 2A-2I show the effects of SOX-18-enforced expression on hematopoietic differentiation of human pluripotent stem cells (hPSCs). FIG. 2A is a diagram of a timeline of hematopoietic differentiation, wherein D represents day of differentiation. Cell types arising after treatment of developing cell cultures set forth horizontal are shown at each developmental day underneath the timeline. FIG. 2B is a diagram of the experimental design. Cells were differentiated following DOX treatment as indicated. Floating HPs were collected on D8 of differentiation and evaluated for phenotype, CFC and lymphoid potential. FIG. 2C shows flow cytometric counter plots illustrating a phenotype of hematopoietic progenitors isolated form D8 differentiation cultures of DOX-induced SOX18 hPSCs. FIG. 2D illustrates percentages of CD34+CD43+ cells and composition of CD34+CD43+ subsets on D8 of differentiation (results are mean ±SD, n=5 for two independent experiments performed in triplicate and duplicate). *p<0.05, **p<0.01 and ***p<0.001, one-way ANOVA, Tukey's multiple comparisons test. FIG. 2E is a graph showing CFC potential of D8 HPs, wherein results shown are mean ±SD, n=2. **p<0.01, two-way ANOVA. FIG. 2F shows Flow cytometric analysis of T cell differentiation induced by DOX on differentiating human pluripotent stem cells (hPSCs). FIG. 2G shows the total number of T cells generated from 104 of D8 CD43+cells and percentages of CD5+CD7+ and CD4+CD8+ cells (results are means ±SD, n=5 experiments). *p<0.05, **p<0.01 ***p<0.001, and ****p<0.0001, one-way ANOVA, Dunnett's multiple comparisons test. FIG. 2H shows flow cytometric analysis of NK cell differentiation induced by DOX on differentiating hPSCs. FIG. 21 is a graphical representation showing the total number of CD56+cells generated from 104 CD43+ cells and the expression of CD94 and CD16 by NK cells (results are means ±SD, n=6 for two independent experiments performed in triplicate). *p<0.05, **p<0.01, ***p<0.001, and ****p<0.0001, one-way ANOVA, Dunnett's multiple comparisons test.

FIGS. 3A-3E show experimental results showing that SOX18 has a limited effect on specification of hemogenic endothelium. FIG. 3A shows flow cytometric results demonstrating expression of arterial markers and Venus in VEC⁺ SOX18 cells on day 4 of differentiation with or without DOX. FIG. 3B and FIG. 3C are graphical representations showing percentages and total number of VEC⁺ cells, DLL4+CXCR4⁺ and VEC+DLL4⁺CXCR4⁻ cells generated from 10,000 hPSCs on day 4 of differentiation, wherein the results are shown as mean ±SD, n=3. ***p<0.001, t-test. FIG. 3D shows flow cytometric results demonstrating expression of arterial markers and Venus in VEC+iSOX18 cells on D4 of differentiation with or without DOX. FIG. 3E and FIG. 3F are graphical representations showing percentages and total number of VEC+ cells, VEC+DLL4+CXCR4+ and VEC+DLL4+CXCR4− cells generated from 10⁴hPSCs on D5 of differentiation with or without DOX (wherein results are shown as the mean ±SD, n=3). ***p<0.001, t-test, and **p<0.01, two-way ANOVA, Sidak's multiple comparisons test.

FIGS. 4A-4F show results of experiments illustrating that SOX18 overexpression affects lymphoid specification from HE cells by shifting the balance of NK and T lymphocyte differentiation. FIG. 4A is a schematic diagram illustrating the experimental design as set forth in Example 3. FIG. 4B is a graphical representation of CFC potential of HP generated from D4 HE after coculture on OP9 or DLL4-0P9 for 5 days with or without DOX, the results expressed as mean ±SD, n=2. **p<0.01 and ****p<0.0001, two-way ANOVA, Tukey's multiple comparisons test. FIG. 4C is a graphical representation of total number of CD4⁺CD8⁺ T cells generated from 10,000 CD43⁺ cells, the results expressed as mean ±SD, n=4. ****p<0.0001, two-way ANOVA, Sidak's multiple comparisons test. FIG. 4D shows flow cytometric results demonstrating the effect of SOX18 overexpression on T cell differentiation from hemogenic endothelium. FIG. 4E is a graphical representation of the total number of CD56⁺ NK cells generated from 10,000 CD43⁺ cells, the results expressed as mean ±SD, n=4. ****p<0.0001, two-way ANOVA, Sidak's multiple comparisons test. FIG. 4F shows flow cytometric results demonstrating the effect of SOX18 overexpression on NK cell differentiation from hemogenic endothelium.

FIGS. 5A-5G illustrate characterization of NK cell potential in iSOX 18 hPSCs. FIG. 5A is a diagram of the experimental design showing DOX treatment schedule and cell subsets analyses as set forth in Example C. FIG. 5B shows flow cytometric results demonstrating NK cell differentiation potential of cell subsets isolated from cultures in the presence and absence of DOX. FIG. 5C is a graphical representation of total number of CD56⁺ NK cells generated from 10,000 cells from the indicated cell subset, the results expressed as mean ±SDs, n=4. ****p<0.0001, one-way ANOVA, Tukey's multiple comparisons test. FIG. 5D is a graphical representation of the total numbers of CD56+cells in cultures from No DOX and DOX2-8 HP after 1- and 4-weeks differentiation in NK cultures (results are means ±SDs, n=10-11). ****p<0.0001, two-way ANOVA, Sidak's multiple comparisons test. FIG. 5E is a graphical representation of a growth curve of CD56+ cells in NK differentiation cultures initiated from D8 CD43+ cells generated in No DOX and DOX2-8-treated cultures. Cell cultures were started with 10⁵CD43+ cells and continued for 5 weeks (results are means ±SDs, n=2). FIG. 5F is a graphical representation of a cytotoxicity assay against K562 targets. Different amount of target cells plated with CD56+cells (effector: target ratio from 1:1 to 5:1) for 4 h (Results are means ±SDs, n=2 experiments). FIG. 5G is a flow cytometric counter plot shows CD107a and interferon gamma (IFNγ) expression by CD56+cells after K562 stimulation. FIG. 5H is a graphical representation of percentages of CD107a+IFNγ− or CD107a-IFNγ+ cells after PMA/ionomycin stimulation. The results are expressed as mean ±SD, *p<0.05, t-test.

FIGS. 6A-6E illustrate molecular profiling of HE, HPs and NK cells generated from iSOX18 hPSCs. FIG. 6A shows flow cytometric results illustrating cell populations isolated for RNAseq analysis. FIG. 6B is a graphical representation showing the number of DEGs in indicated cell populations. FIG. 6C is a heatmap showing differentially expressed genes in D4 HE P1 population from cultures with and without DOX. FIG. 6D shows bar plots illustrating the presence of significantly enriched KEGG pathways in selected differential expression comparisons. The y axis shows the enriched pathway categories and x axis shows the number of significantly genes in each pathway. FIG. 6E is a bar graph that shows significantly enriched KEGG pathways in selected differential expression comparisons. The y axis shows the enriched pathway categories and x axis shows the number of significantly enriched genes in each pathway. FIG. 6F shows the fold changes of selected genes in the two KEGG pathways that are significantly enriched in D8 P1 DOX+ cells over D8 P1 No DOX cells.

FIGS. 7A-7D illustrate generation of DOX-inducible SOX18 in H1 hESC line. FIG. 7A shows a flow Cytometric analysis in undifferentiated iSOX18 cells cultured with or without DOX. FIG. 7B are fluorescent images show the expression of Venus reporter in undifferentiated iSOX18 cells cultured with or without DOX. Scale bars are 200uM. FIG. 7C shows a qRT-PCR analysis shows SOX18 expression in D4 HE generated in iSOX18 hPSCs differentiation cultures with or without DOX. Results are mean +− SD (N=9). ****p<0.0001, t-test. FIG. 7D is a western blot showing upregulation of SOX18 expression in undifferentiated iSOX18 cells 24 hours after DOX treatment and D5 differentiated iSOX18 hPSCs treated with DOX at D2-D5.

FIGS. 8A-8C provide results of an in-depth analysis of D8 CD43⁺ subsets collected from cultures without and with D2-8 DOX using t-distributed stochastic neighbor embedding algorithm (t-SNE). FIG. 8A is an overlayed t-SNE plot of No DOX and DOX2-8 CD43+ subsets at D8. Each dot represents one cell. FIG. 8B shows that No DOX or DOX2-8 samples created single tSNE with single surface marker in each plot. Expression levels of marker defines with a continuous grayscale scale. FIG. 8C shows tSNE maps generated for the No DOX or DOX2-8 subsets. Each shade indicates No DOX or DOX2-8 cells at D8 CD43⁺. Each dot represents one cell and cells are shaded according to their assigned subsets. Cell populations were defined by the manual gating strategy.

FIGS. 9A-9C show the effect of SOX18 overexpression on development of megakaryocytes and hemangioblasts (HBs). FIG. 9A show counter plots demonstrating expression megakaryocytic markers in megakaryocyte differentiation cultures initiated using CD43+cells collected from No DOX and DOX2-8 cultures at D8. FIG. 9B is a graphical representation of the percentage of megakaryocytic cells generated in these cultures. The results are presented as mean ±SD, n=3. ** p<0.01 and ***p<0.001, t-test. FIG. 9C is a graphical representation of the HB-CFC potential of iSOX18 cells. Cells were collected from D3 differentiation without DOX or with D2-3 DOX treatment. The graph shows number of HB colonies per 10,000 cells collected on D3 of differentiation. The results are presented as the mean ±SD, n=2. **p<0.01, t-test.

FIGS. 10A-10D illustrate the impact of SOX18 overexpression on cell cycle and apoptosis. FIG. 10A shows an analysis of gene enrichment in GO “Apoptosis”. FIG. 10B shows the analysis of gene enrichment in GO “Regulation of Cell Population Proliferation” gene sets in indicated cell subsets in DOX-treated and untreated cultures. Shaded areas indicates significant enrichment in DOX-treated over untreated cultures. FIG. 10C show representative dot plots of flow cytometric analysis of cell cycle in the D5 HE in DOX and No DOX iSOX18 hPSC cultures. FIG. 10D is a bar graph showing the means +− SD (D) of duplicated experiments of the flow cytometric analysis of cell cycle in the D5 HE in DOX and No DOX iSOX18 hPSC cultures. ***p<0.001 , and ****p<0.0001, one-way ANOVA Dunnett's multiple comparisons test.

### DETAILED DESCRIPTION OF THE DISCLOSURE

The present disclosure is based, at least in part, on experimental demonstration that forced SOX18 expression in cells in the hematopoietic lineage preferentially promotes formation of progenitors of natural killer cells and megakaryocytes.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, for example, are expressly enumerated in this disclosure. These are only examples of what is specifically intended, and all possible combinations of numerical values between and including the lowest value and the highest value enumerated are to be considered to be expressly stated in this disclosure.

The term “contacting” includes the physical contact of at least one substance to another substance.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

The terms “express” or “expression” refer to transcription and translation of a nucleic acid coding sequence resulting in production of the encoded polypeptide. “Express” or “expression” also refers to antigens that are expressed on cell surfaces.

Unless otherwise defined, all technical terms used herein have the same meaning as commonly understood by a person of ordinary skill in the art to which this disclosure belongs.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The human subject can be of any age (e.g., an infant, child, or adult).

The term “construct” refers to an artificially-designed segment of DNA that can be used to incorporate genetic material into a target cell (e.g., an hPSC).

The term “sequence identity” refers to the number of identical or similar nucleotide bases on a comparison between a test and reference oligonucleotide or nucleotide sequence. Sequence identity can be determined by sequence alignment of a first nucleic acid sequence to identify regions of similarity or identity to second nucleic acid sequence. As described herein, sequence identity is generally determined by alignment to identify identical residues. Matches, mismatches, and gaps can be identified between compared sequences by techniques known in the art. Alternatively, sequence identity can be determined without taking into account gaps as the number of identical positions/length of the total aligned sequence ×100. In one embodiment, the term “at least 90% sequence identity to” refers to percent identities from 90 to 100%, relative to the reference nucleotide sequence. Identity at a level of 90% or more is indicative of the fact that, assuming for exemplary purposes a test and reference polynucleotide sequence length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

Any appropriate method can be used to detect expression of biological markers characteristic of cell types described herein. For example, the presence or absence of one or more biological markers can be detected using, for example, RNA sequencing (e.g., RNA- seq), immunohistochemistry, polymerase chain reaction, quantitative real time PCR (qRT-PCR), or other technique that detects or measures gene expression. RNA-seq is a high-throughput sequencing technology that provides a genome-wide assessment of the RNA content of an organism, tissue, or cell. Alternatively, or additionally, one can detect the presence or absence of, or measure the level of, one or more biological markers of HPCs using, for example, Fluorescence in situ Hybridization (FISH; see W098/45479 published October 1998), Southern blotting, Northern blotting, or polymerase chain reaction (PCR) techniques, such as qRT-PCR. In exemplary embodiments, a cell population obtained according to a method provided herein is evaluated for expression (or the absence thereof) of biological markers of HPCs such as CD34, CD45, CD43, and CD90. Quantitative methods for evaluating expression of markers at the protein level in cell populations are also known in the art.

The term “genetically engineered” as used herein refers to cells that have been manipulated using biotechnology to change the genetic makeup of the cells, including the transfer of genes within and across species boundaries to produce improved or non-naturally occurring cells. A human pluripotent stem cell, hemogenic endothelium, megakaryocytes or NK cells that contains an exogenous, recombinant, synthetic, and/or otherwise modified polynucleotide is considered to be a genetically engineered cell and, thus, non-naturally occurring relative to any naturally occurring counterpart. In some cases, genetically engineered cells contain one or more recombinant nucleic acids. In other cases, genetically engineered cells contain one or more synthetic or genetically engineered nucleic acids (e.g., a nucleic acid containing at least one artificially created insertion, deletion, inversion, or substitution relative to the sequence found in its naturally occurring counterpart). Procedures for producing genetically engineered cells are generally known in the art, for example, as described in Sambrook et al, Molecular Cloning, A Laboratory Manual (Fourth Edition), Cold Spring Harbor Press, Cold Spring Harbor, N.Y. (2012) and Doudna et al., CRISPR-Cas, A Laboratory Manual, Cold Spring Harbor Press, Cold Spring Harbor, N.Y. (2016).

A genetically engineered cell can be a cell that has been modified using a gene editing technique. Gene editing refers to a type of genetic engineering in which DNA is inserted, deleted, modified, or replaced in the genome of a living cell. In contrast to other genetic engineering techniques that can randomly insert genetic material into a host genome, gene editing can target the insertions to site specific locations (e.g., AAVS1 alleles). Examples of gene editing techniques including, but are not limited to, restriction enzymes, zinc finger nucleases, TALENs, and CRISPR-Cas9.

A genetically engineered cell can be a stem cell (e.g., a human pluripotent stem cell) or any of their differentiated progeny cells (e.g., mesoderm cells, hemangioblast cells, hemogenic endothelium cells, hematopoietic progenitor cells, megakaryocytes and NK cells) that have been modified to express. Any of the cells described herein can be genetically engineered. In some embodiments, a genetically engineered cell refers to a cell that is differentiated from a cell that has been genetically engineered.

The term “tumor cell” as used herein refers to abnormal cells that divide continuously. In some embodiments, the tumor cell is a solid tumor cell. A solid tumor is an abnormal mass of cells that typically does not contain cysts or a liquid area. Examples of solid tumors include, but are not limited to, sarcomas and carcinomas. Cancers of the blood (e.g., leukemias) typically do not form solid tumors. In some embodiments, the “tumor cell” is not a blood cancer cell. “Tumor cells” as used herein refers to a group of tumor cells and/or a single tumor cell.

Human pluripotent stem cells (hPSCs), either embryonic or induced, provide access to the earliest stages of human development and offer a platform on which to derive a large number of hematopoietic progenitor cells or blood cells for cellular therapy and tissue engineering. Accordingly, the methods provided herein can comprise differentiating human pluripotent stem cells under conditions that promote differentiation of mesodermal cells (e.g., arterial endothelial cells) into hematopoietic progenitor cells into megakaryocytes and NK cells.

Methods of differentiating hPSCs into progenitor mesoderm, hemogenic endothelium and hematopoietic progenitors are known in the art. In exemplary embodiments the culture medium to be used in any of the above-described differentiation methods comprises an IF9S medium, as described herein. In one embodiment, the IF9S medium to be used is the IF9S medium having the formulation set forth in Table 2. In some embodiments, any of the above-referenced cells (e.g., human pluripotent stem cells) are cultured on Tenascin C. In some embodiments, any of the referenced cells are seeded on a substrate treated with an amount of Tenascin-C sufficient to adhere 10,000 cells/cm² to the substrate. In some embodiments, the Tenascin-C to be used is human Tenascin C. In some embodiments, the substrate is treated with Tenascin C at a concentration of at least about 0.25 μg/cm² to 1 μg/cm², e.g., 0.4 μg/cm², 0.5 μg/cm², 0.7 μg/cm², 0.8 μg/cm², or another concentration from at least about 0.25 μg/cm² to 1 μg/cm². In a preferred embodiment, the cells are cultured on plates coated with Collagen IV, as described in Uenishi et al., 2014, Stem Cell Reports 3: 1073-1084 and U.S. Pat. No. 9,938.499.

In some embodiments, in the cell culture medium to be used in the differentiation methods set forth herein, the concentration of: BMP4 is about 50 ng/ml to about 250 mg/ml; Activin A is about 10 ng/ml to about 15 ng/ml; FGF2 is about 10 ng/ml to about 50 ng/ml; LiCl is about 1 mM to about 2 mM; VEGF is about 20 ng/ml to about 50 ng/ml; SCF is about 50 ng/ml to about 100 ng/ml; TPO is about 50 ng/ml to about 100 ng/ml; IL-6 is about 50 ng/ml to about 100 ng/ml, and IL-3 is about 5 ng/ml to about 15 ng/ml.

In some embodiments, any of the above-referenced cells are cultured in a xeno-free cell culture medium. Of central importance for clinical therapies is the absence of xenogenic materials in the derived cell populations, i.e., no non-human cells, cell fragments, sera, proteins, and the like. Preferably, the present invention arrives at xenogen-free differentiated cells by use of Tenascin C or Collagen IV as a platform, which essentially replaces contact with OP9 cells used in earlier differentiation systems. In addition, the media disclosed herein are chemically defined and, in some embodiments, are made xeno-free, and incorporate human proteins, which can be produced using recombinant technology or derived from placenta or other human tissues in lieu of animal-derived proteins. In some embodiments, all proteins added to the medium are recombinant proteins.

As used herein, the term “mesoderm cell” refers to a cell having mesoderm-specific gene expression, capable of differentiating into a mesodermal lineage such as bone, muscle such as cardiac muscle, skeletal muscle, and smooth muscle (e.g., of the gut), connective tissue such as the dermis and cartilage, kidneys, the urogenital system, blood or hematopoietic cells, heart, and vasculature. Mesoderm-specific biomarkers include Brachyury (7). Culturing can take place on any appropriate surface (e.g., in two-dimensional or three-dimensional culture).

Medium and substrate conditions for culturing pluripotent stem cells, as used in the methods described herein, are well known in the art. In some cases, pluripotent stem cells to be differentiated according to the methods disclosed herein are cultured in mTESR-1 medium (StemCell Technologies, Inc., Vancouver, British Columbia.), E8 medium, or Essential 8 medium (Life Technologies, Inc.) on a MATRIGEL™ substrate (BD Biosciences, NJ) or Vitronectin (Life Technologies) according to the manufacturer's protocol.

As used herein, the term “albumin-free conditions” indicates that the culture medium used contains no added albumin in any form including, without limitation, Bovine Serum Albumin (BSA), any form of recombinant albumin, or any other animal albumin.

As used herein, the terms “chemically-defined medium” and “chemically-defined culture medium” also refer to a culture medium containing formulations of fully disclosed or identifiable ingredients, the precise quantities of which are known or identifiable and can be controlled individually. As such, a culture medium is not chemically-defined if (1) the chemical and structural identity of all medium ingredients is not known, (2) the medium contains unknown quantities of any ingredients, or (3) both. Standardizing culture conditions by using a chemically-defined culture medium minimizes the potential for lot-to-lot or batch-to-batch variations in materials to which the cells are exposed during cell culture. Accordingly, the effects of various differentiation factors are more predictable when added to cells and tissues cultured under chemically-defined conditions.

As used herein, the term “serum-free” refers to cell culture materials that do not contain serum or serum replacement, or that contains essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. “Serum free” also refers to culture components free of serum obtained from animal (e.g., fetal bovine) blood or animal-derived materials, which is important to reduce or eliminate the potential for cross-species viral or prion transmission. For avoidance of doubt, serum-containing medium is not chemically-defined.

Human pluripotent stem cells (e.g., human ESCs or iPS cells) can be cultured in the absence of a feeder layer (e.g., a fibroblast feeder layer), a conditioned medium, or a culture medium comprising poorly defined or undefined components. As used herein, “feeder-free” refers to culture conditions that are substantially free of a cell feeder layer. Cells grown under feeder-free conditions can be grown on a substrate, such as a chemically-defined substrate, and/or grown as an adherent culture. Suitable chemically-defined substrates include vitronectin.

A method of producing a hematopoietic progenitor cell can comprise culturing human pluripotent stem cells in a serum-free, albumin-free, chemically-defined culture medium that promotes differentiation to mesoderm. In this manner, pluripotent stem cell-derived mesodermal cells are differentiated according to the HPC differentiation methods provided herein, thus producing pluripotent stem cell-derived HPCs.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., Science 282:1145-1147 (1998). These cells can express Oct-4, SSEA-3, SSEA-4, TRA-1-60, and TRA-1-81. Pluripotent stem cells appear as compact colonies comprising cells having a high nucleus to cytoplasm ratio and prominent nucleolus. ESCs are commercially available from sources such as WiCell Research Institute (Madison, WI.).

As used herein, “induced pluripotent stem cells” or “iPS cells” refers to pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli, and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60, or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

As set forth herein, NK cells and megakaryocytes can be preferentially produced, compared with T cells, by inducing expression in SOX18 in progenitor cells. Generally, preferential differentiation can be achieved by overexpressing SOX18 at a specific time (i.e., starting at day 2) and for a defined period (i.e., 2-8 days) during differentiation. SOX18 is a member of the Sry-related high mobility group domain (SOX) family of transcription factors and is key developmental regulator of endothelial and hematopoietic lineages. In the methods of the present invention, differentiating hPSCs are forced to overexpress SOX18 during the mesoderm differentiation by introducing an inducible SOX18 transgene into the population of progenitor cells.

The SOX18 transgene used with the present invention can comprise any nucleic acid sequence encoding the SOX18 protein. For example, the SOX18 transgene can be obtained by amplifying the SOX18 gene sequence from the genomic locus in human cells or by amplifying SOX18 mRNA from hPSCs differentiated into endothelial and blood cells and converting it into cDNA. Alternatively, genomic DNA or cDNA clones can be obtained commercially (e.g., from Sino Biological, Origene, or IDT). In some embodiments, the transgene comprises SEQ ID NO.:16, a cDNA sequence encoding the human SOX18 protein (having an amino acid sequence set forth in SEQ ID NO: 17).

In some embodiments, the SOX18 transgene further comprises a vector sequence that can be used to drive the expression of the SOX18 transgene within the cells. In these embodiments, the transgene is introduced by into the population of hPSCs by transducing the cells with said vector. As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

Vectors suitable for use with the present invention comprise a nucleotide sequence encoding a SOX18 transgene and a heterogeneous sequence necessary for proper propagation of the vector and expression of the encoded polypeptide. The heterogeneous sequence (i.e., sequence from a different species than the transgene) can comprise a heterologous promoter or heterologous transcriptional regulatory region that allows for expression of the polypeptide. Suitable vectors for the expression of the SOX18 transgene include plasmids and viral vectors. In a preferred embodiment, the vector comprises heterologous sequence that allows the transient and/or Inducible expression of the encoded SOX18 protein.

In some embodiments, the vector includes a transposase system, such as the PiggyBac transposon system (see Examples). The PiggyBac transposon is a TTAA-specific mobile genetic element that efficiently transposes between vectors and chromosomes via a “cut and paste” mechanism. PiggyBac transposase recognizes transposon-specific inverted terminal repeat sequences (ITRs) and moves the intervening contents to a TTAA insertion site in a chromosome or another vector. Thus, inserting a gene of interest between two ITRs in a transposon vector allows one to efficiently insert the gene into a target genome. Other suitable transposase systems for use with the present invention include, for example, Sleeping Beauty.

In other embodiments, the vector is a plasmid, a viral vector, a cosmids, or an artificial chromosome. Suitable plasmids include, for example, E. coli cloning vectors. Many suitable viral vectors are known in the art and include, but are not limited to, an adenovirus vector; an adeno-associated virus vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector. In one particular embodiment, the vector comprises SEQ ID NO:15.

In some embodiments, the vector is an expression vector that comprises a promoter that drives the expression of the SOX18 transgene, preferably transient or inducible expression of the SOX18 transgene. As used herein, the term “promoter” refers to a DNA regulatory region capable of binding RNA polymerase in a cell and initiating transcription of a coding sequence. Although promoters are most commonly found immediately upstream of a coding sequence, they can also be found downstream of or within the coding sequence. Promoters can be derived in their entirety from a native gene or can be composed of multiple elements, including elements derived from promoters found in nature or elements comprising synthetic DNA sequences. It is understood by those skilled in the art that different promoters can direct the expression of a gene in different tissues or cell types, at different stages of development, or in response to different environmental conditions. Preferably, the promoters used with the present invention are inducible promoters. An “inducible promoter” is a promoter that is activated (i.e., initiates transcription) only in the presence of a particular molecule. Inducible promoters allow tight control the expression of a transgene within cells. Many suitable inducible expression systems are known in the art and include, for example, Tet-On gene expression systems that allow one to induce the expression of a gene by administering tetracycline (Tc) or tetracycline-derivatives like doxycycline (DOX). Suitable Tet-On systems for use with the present invention include, without limitation, Tet-On Advanced and Tet-On 3G. Tet-On systems utilize several promoters, including both minimal promoters (e.g., CMV) flanked by a tetracycline response element (TRE) and engineered Tet-inducible promoters (e.g., TRE2 and TREtight). For instance, in the Examples, the SOX18 transgene is inserted (i.e., via conventional cloning methods) downstream of the doxycycline-inducible TREtight promoter within a vector. This vector was introduced into the hPSCs, allowing the inventors to induce expression of the SOX18 transgene at the desired stage of differentiation by adding doxycycline to the cell culture to activate expression from the TREtight promoter. Those of skill in the art are aware of many additional inducible gene expression systems, including both chemical-inducible and temperature-inducible systems. Other suitable inducible gene expression systems for use with the present invention include, without limitation, the glucocorticoid-responsive mouse mammary tumor virus promoter (MMTVprom), the tamoxifen-responsive hormone-binding domain of the estrogen receptor (ERTAM), the ecdysone-inducible promoter (EcP), heat shock inducible promoters (e.g., Hsp70 or Hsp90- derived promoters), and the T7 promoter/T7 RNA polymerase system (T7P). The SOX18 transgene can be introduced into the hPSCs using any suitable method, for example by transfection or transduction. In one embodiment, the transgene is introduced by transducing the hPSCs with a vector comprising the SOX18 transgene. In another embodiment, the hPSCs are transduced with an exogenous SOX18 modified mRNA (mmRNA). In yet another embodiment, the hPSCs are transduced with the SOX18 protein. Typically, mmRNAs comprise (i) a 5′ synthetic cap for enhanced translation; (ii) modified nucleotides that confer RNAse resistance and an attenuated cellular interferon response, which would otherwise greatly reduce translational efficiency; and (iii) a 3′ poly-A tail.

As provided herein, SOX18 can be enforced in progenitor cells by introduction of an inducible recombinant genetic construct encoding human SOX18 having a nucleotide sequence set forth herein. As used herein, the term “enforced” when used with regard to SOX18 expression in a progenitor cell for NK cells or megakaryocytes produced according to the methods disclosed herein can be understood by the skilled worker to mean that SOX18 expression in the cell is increased over endogenous SOX18 expression in the cell by introduction therein of an exogenous SOX18-encoding construct wherein SOX18 expression is controlled by an inducible promoter. Endogenous SOX18 can be enforced, inter alia, using reagents and methods known in the art, including but not limited to disclosure in U.S. Patent Application Nos. U.S. 2018/0010124 and 2018/0142207, the disclosures of which are expressly incorporated by reference herein. Such techniques and methodologies can comprise enforced expression of SOX18 in said cells.

As used herein, the term “forced expression ” refers to inducing an increase in the level of a protein of interest (e.g., a transcription factor) in a population of host cells, e.g., hPSCs. Forced expression can include one or more of the following in any combination: introducing exogenous nucleic acids encoding the protein of interest (e.g., by viral transduction, plasmid expression vector transfection, or modified mRNA transfection); protein transduction; genomic modification of a host cell, e.g., replacing a promoter to increase the expression of an endogenous (native) gene; and contacting host cells with a small molecule that induces increased expression of an endogenous protein.

### Pharmaceutical Formulations and Methods of Treatment

Administration of a therapeutically effective amount of NK cells or megakaryocytes provided herein into the recipient subject is generally effected using methods well known in the art, and usually involves directly injecting or otherwise introducing a therapeutically effective dose of NK progenitor cells into the subject using clinical tools known to those skilled in the art (e.g., U.S. Pat. Nos. 6,447,765; 6,383,481; 6,143,292; and 6,326,198). For example, introduction of NK cells or megakaryocytes of the present invention can be injected locally or systemically via intravascular administration, such as intravenous, intramuscular, or intra-arterial administration, intraperitoneal administration, and the like. Cells can be injected into an infusion bag (e.g., Fenwal infusion bag (Fenwal, Inc.)) using sterile syringes or other sterile transfer mechanisms. The cells can then be immediately infused via IV administration over a period of time, such as 15 minutes, into a free flow IV line into the patient. In some embodiments, additional reagents such as buffers or salts are provided to the recipient subject concurrently with the cells.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques preformed as exemplified herein.

### Materials and Methods

Cell Culture. Wild type H1 and human pluripotent stem cells (hPSCs) were obtained from WiCell (Madison, WI).

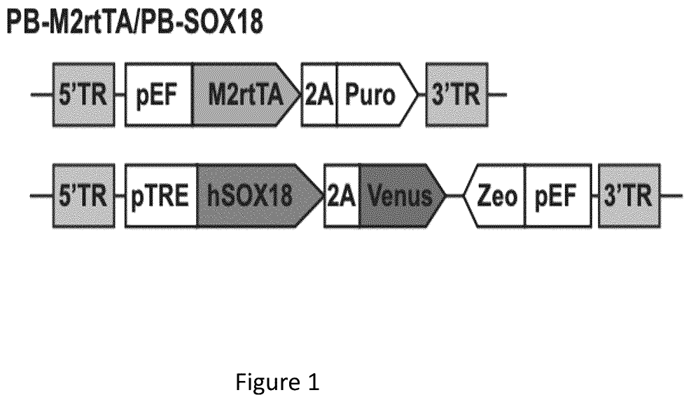

Construction of vectors, generation, and validation of iSOX18 hPSC line. A doxycycline (DOX)-inducible SOX18 H1 hESC line was generated using the PiggyBac system disclosed in Park et al., 2018, Curr. Protoc. Stem Cell Biol. 47: e63 and illustrated in FIG. 1. Human SOX18 CDS was cloned into PiggyBac transposon vector (Transposagen) downstream of TREtight promoter of pTRE-P2A-Venus-rpEFla-Zeo plasmid, and co transfected with pEF 1α-M2rtTA-T2A-Puro and transposase plasmid into H1 cells using human stem cell nucleofector kit 2 (Lonza). Cells were selected in Zeocin (0.5 μg/ml, Thermofisher) and Puromycin (0.5 μg/ml, Sigma) for 10 days and resistant clones screened by Venus expression with DOX (Sigma) treatment.

hPSC maintenance and hematopoietic differentiation. hPSCs were maintained and passaged on Matrigel in mTeSR1 media (WiCell). Hematopoietic differentiation was performed on collagen IV (ColIV)-coated plate in chemically defined serum-free medium as previously described as disclosed in Uenishi et al., 2014, Stem Cell Reports 3: 1073-1084. The iSOX18 H1 line from hPSCs (H1 hESC line from WiCell) was maintained and passaged on Matrigel in mTeSR1 media (WiCell). The cell lines were differentiated on a collagen IV (ColIV)-coated plate. Briefly, to initiate differentiation, cells were plated at 5,000 cells/cm² onto 6 well plates with E8 media and 10 μM Rock inhibitor (Y-27632, Cayman Chemicals). This media was changed the following day to IF9S media with 50 ng/ml FGF2 (PeproTech), 50 ng/ml BMP4 (PeproTech), 15 ng/ml Activin A (PeproTech), and 2 mM LiCl (Sigma), and cells were cultured in hypoxia (5% CO2, 5% O₂). On day 2, the media was changed to IF9S media with 50 ng/ml FGF2, 50 ng/ml VEGF (PeproTech), and 2.5 μM TGF-f3 inhibitor (SB-431542, Cayman), and cells were cultured in hypoxia (5% CO₂, 5% O₂). On days 4 and 6, the media was changed to IF9S media with 50 ng/ml FGF2, 50 ng/ml VEGF, 50 ng/ml TPO (PeproTech), 50 ng/ml IL-6 (PeproTech), 20 ng/ml SCF (PeproTech), and 10 ng/ml IL-3 (PeproTech), and cells were cultured in normoxia (20% CO₂, 5% O₂).

Isolation and culture of hemogenic endothelium and hematopoietic progenitors. CD31⁺ cells from day 4 (D4) cultures of iSOX18 cells were isolated by magnetic activated cell sorting (MACS) using CD31 antibodies. In D4 differentiation cultures almost all CD31⁺ cells co-express VE-cadherin. Selected cells were plated on OP9 or DLL4-0P9 in 10% α-MEM with 10% FBS (Hyclone) with TPO, SCF (50 ng/ml), IL-6 (20 ng/ml), IL-3 and FLT3L (10 ng/ml). Medium was changed on the following day and extra medium was added on the 3rd day of OP9 co-culture. After 5 days in secondary culture, cells were collected and assessed for colony-forming cells (CFCs), T, and NK potential. Hematopoietic precursor cells (HPs) generated from iSOX18 cells were collected at day 8 (D8) of differentiation. Four different subsets of CD34+CD43+ population CD235a/CD41a-CD45− (P1 subset), CD235a/CD41a+CD45− (P2), CD235a/CD41a+CD45+ (P3) and CD235a/CD41a-CD45+ (P4) were isolated using a MA900 cell sorter (Sony Biotechnology) and cultured on OP9-DLL4 in NK cell differentiation conditions.

Hemangioblast (HB)-CFC and hematopoietic CFC assay. HB-CFC were detected as described in Vodyanik et al., 2010, A mesoderm-derived precursor for mesenchymal stem and endothelial cells, Cell Stem Cell 7: 718-729. Hematopoietic CFCs were detected using serum containing H4435 MethoCult (Stem Cell Technologies). HB-CFCs were detected using a semisolid colony-forming serum-free medium (CF-SFM) containing 40% ES-Cult M3120 methylcellulose (2.5% solution in IMDM, Stem Cell Technologies), 25% StemSpan serum-free expansion medium (SFEM, Stem Cell Technologies), 25% human endothelial serum-free medium (ESFM, ThermoFisher), 10% BIT 9500 supplement (Stem Cell Technologies), GlutaMAX (1/100 dilution, ThermoFisher), Ex-Cyte (1/1000 dilution, Millipore), 100 mM MTG, 50 mg/mL ascorbic acid and 20 ng/mL FGF (Peprotech)

Megakaryocyte differentiation. Floating hematopoietic cells from iSOX18 hPSC cultures were collected at D8 of differentiation and cultured in StemSpan serum-free expansion medium (SFEM, Stem Cell Technologies) with 20 ng/ml SCF, TPO, and IL-11 on an ultra-low attachment 6- well plate for 5 days. Fresh media (2 mL) was added every 2 days. All cytokines were purchased from PeproTech.

NK-cell differentiation. For NK cell differentiation, floating hematopoietic cells from D8 primary cultures or day 4+5 secondary OP9/0P9-DLL4-cocultures were cultured in α-MEM (Invitrogen) with 20% FBS (Hyclone), 25 ng/ml SCF, 5 ng/ml FLT3L, IL-3 and IL-7 and 10 ng/ml IL-15 (PeproTech) on OP9-DLL4 for 5 days. Cells were then cultured in the same media without IL-3 for 3-4 weeks. Cells were passaged weekly onto fresh DLL4-0P9 cells and analyzed by flow cytometry for NK cell surface markers after 3-4 weeks.

Functional analysis of NK cells. To assess cytotoxicity, CD56⁺ cells were isolated using a MA900 cell sorter and incubated with K562-GFP target cells for 4 hours at 37° C., at effector:target (E:T) ratios of 1:1, 2.5:1, and 5:1, in 96-well plates. Cells were collected in FACS buffer and stained using 7-Aminoactinomycin D (7-AAD) and Annexin V (BD). Specific killing was calculated by subtracting spontaneous K562 death (7-AAD+ cells in no effector control). Production of IFNγ and CD107a expression in isolated CD56⁺ cells was assessed after incubation with Phorbol 12-myristate 13-acetate (PMA) and ionomyocin (1:500) (BioLegend) for 4 hours. Brefeldin A (1:1000; ThermoFisher) was added at the beginning of the stimulation. Cells were washed with FACS buffer and stained with Live/dead violet 540 (TONBO Bioscience) with CD107a antibody (BD). Cells were treated with fixation/permeabilization buffer (eBioscience) and stained for intracellular IFNγ (BD).

T-cell differentiation. For T cell differentiation, floating hematopoietic cells from D8 of primary differentiation cultures or day 4+5 secondary OP9/0P9-DLL4-cocultures were cultured in a-MEM (Invitrogen) with 20% FBS (Hyclone), 10 ng/ml SCF, 5 ng/ml FLT3L and IL-7 (PeproTech) on OP9-DLL4 for 3 weeks. Cells were passaged weekly onto fresh OP9-DLL4 cells. Cells were analyzed by flow cytometry for T cell surface markers after 21 days. All cytokines were purchased from PeproTech.

Flow cytometry and t-distributed stochastic neighbor embedding algorithm (tSNE) analysis. Flow cytometric analysis was performed using antibodies listed in Table 1 with MACSQuant Analyzer 10 (Miltenyi Biotech) and FlowJo software (FlowJo LLC). For tSNE analysis, individual DOX2-8 and No DOX fcs files were imported into FlowJo to exclude doublets, debris, and dead cells. A subset or 11,000 CD43⁺ cells were selected for each sample and concatenated. To generate a tSNE map, the concatenated data were analyzed with the parameters 30 perplexity, 550 iteration number and 1540 learning rate (Eta). Concatenated cells were divided manually into No DOX or DOX2-8 and cell subsets were defined by the manual gating. First, cells CD34⁺ or CD34⁻ were gated and then gated into CD235a/CD41a^{hi}CD45⁻, CD235a/CD41a^{med}CD45⁻,CD235a⁻CD41a⁻CD45−, CD235a/CD41a⁻CD45⁺, or CD235a/CD41a⁺CD45⁺ cells.

Apoptosis and cell cycle analysis. Apoptosis was detected by flow cytometry using Annexin V (BD). For cell-cycle analysis, D5 cells were incubated in culture medium with bromodeoxyuridine (BrdU) (10 μM, BD Pharmingen) for 2 hours and stained with antibodies. For BrdU detection, the BrdU flow kit with 7 AAD was used and performed per the manufacturer's instructions. Fluorescent reagents used for analysis, cell viability, apoptosis, and proliferation are listed in Table 2.

Real Time Quantitative Polymerase Chain Reaction (qPCR). RNA was extracted from D4 CD31⁺ cells isolated from control and DOX-treated cultures of iSOX18 of iSOX173 hPSCs using the RNeasy Plus Micro Kit (QIAGEN). RNA was reverse transcribed into cDNA using random hexamer primers (QIAGEN) with SMART MMLV reverse transcriptase (TaKaRa). qPCR was conducted using TB Green Advantage qPCR Premix (TaKaRa). RPL13A was used as the reference gene to normalize the data. Primer sequences are listed in Table 2.

RNA-Seq data processing and analysis. One hundred nanograms of total RNA was used to prepare sequencing libraries following the Ligation Mediated Sequencing (LM-Seq) protocol (see, Gandillet et al., 2009, Blood 114: 4813-422) and quantified with a Qubit fluorometer (Life Technologies). Final cDNA libraries were quantitated with the Quant-iT PicoGreen Assay Kit (ThermoFisher Scientific), multiplexed, loaded at a final concentration of 1 nM or 2.5 nM, and sequenced as single reads on the NextSeq 2000 (Illumina), respectively. RNA-seq reads were aligned by STAR (version 2.5.2b) to the human genome (version hg38) with GENCODE basic gene annotations (version 38). Gene expression levels were quantified by RSEM (version 1.3.0), and differential expression was analyzed by edgeR (version 3.34.1). A differentially expressed gene was required to have at least two-fold changes and an adjusted p-value <0.05. Gene set enrichment analysis was performed by fgsea (version 1.18.0) with KEGG gene sets from the Molecular Signatures Database (version 7.1). RNA-seq data has been deposited to GEO with the accession code GSE195670 and a review token as yfefeuuwtvcfncx.

Western Blot. For Western Blot experiment, iSOX18 hPSCs were cultured with (2 μg/ml) and without DOX for 24 hours and harvested. In a similar manner, total cells from iSOX18 hPSC differentiation cultures with (D2-D5) and without DOX were collected at day 5 of differentiation. The cells were lysed using Pierce IP lysis buffer with Pierce protease inhibitors. For cell lysate analysis, protein levels were quantified using the Pierce BCA Assay kit (Thermo Fisher, Waltham, MA) and normalized to 8μg of total protein (depending on the individual blot) prior to running on pre-cast 4-12% gradient SDS-PAGE gels and subsequent transfer to PVDF membranes using Bio-Rad Trans-Blot Turbo Transfer System. The membrane was blocked with 5% BSA (Fisher Scientific, BP1600-100) and 5% Difco Skim Milk (BD, 232100) in TBST (1%) for human SOX18 antibody (R&D Systems, 1:1000) and anti-GAPDH (Santa Cruz Biotechnology, 1:5000) probing respectively. The membranes were incubated with primary antibodies overnight at 4° C. after blocking with mild agitation. The membranes were blotted with their corresponding HRP-linked secondary antibodies at room temperature for one hour. The antibodies were diluted in 1% BSA and 1% milk in TBST for SOX18 and GAPDH detection respectively. 1% TBST was used to wash the membranes for three times at 5 minutes intervals. Sheep and rabbit HRP-linked secondary antibodies were purchased from R&D Systems and Santa Cruz Biotechnology. Images were collected using Bio-Rad ChemiDoc XRS+.

Statistical analysis. Data were analyzed using GraphPad Prism version 9 (GraphPad Software Inc.) and Microsoft Excel (Microsoft Corporation). Tests for statistical significance are listed with each experiment; these included two-sided Student's t-tests for paired analyses and one-way ANOVAs, and two-way ANOVAs for experiments with multiple comparisons of variables or grouped variables, accompanied by the Tukey and Sidak post-hoc test, as inferred to be most appropriate by the software.

### Example 1

### SOX18-Enforced Expression Enhances Production of Erythromyeloid Progenitors

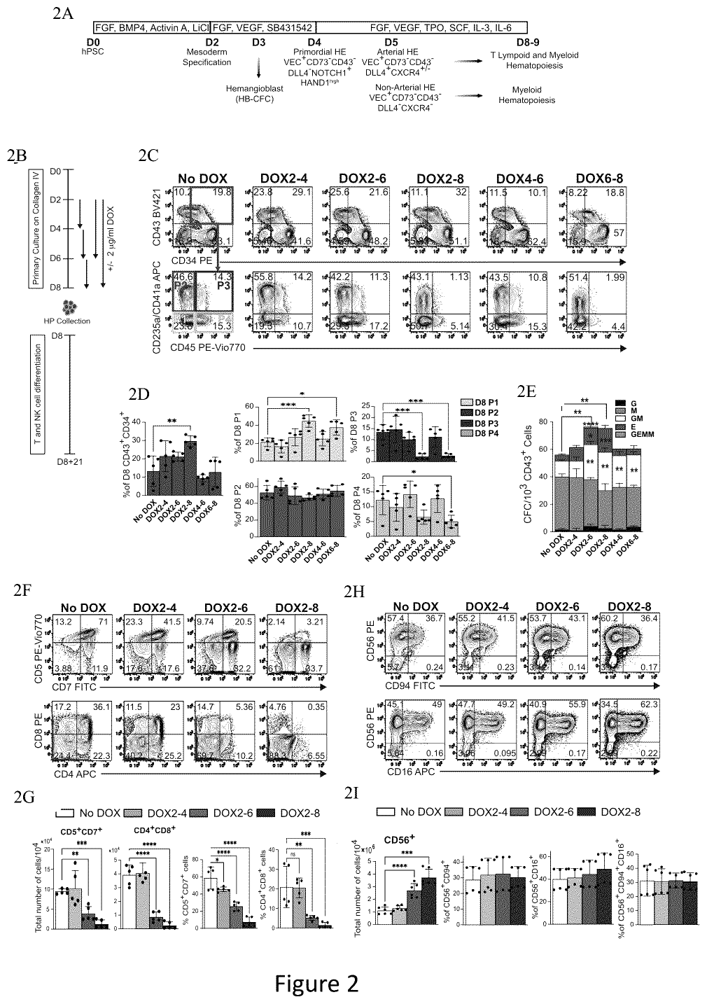

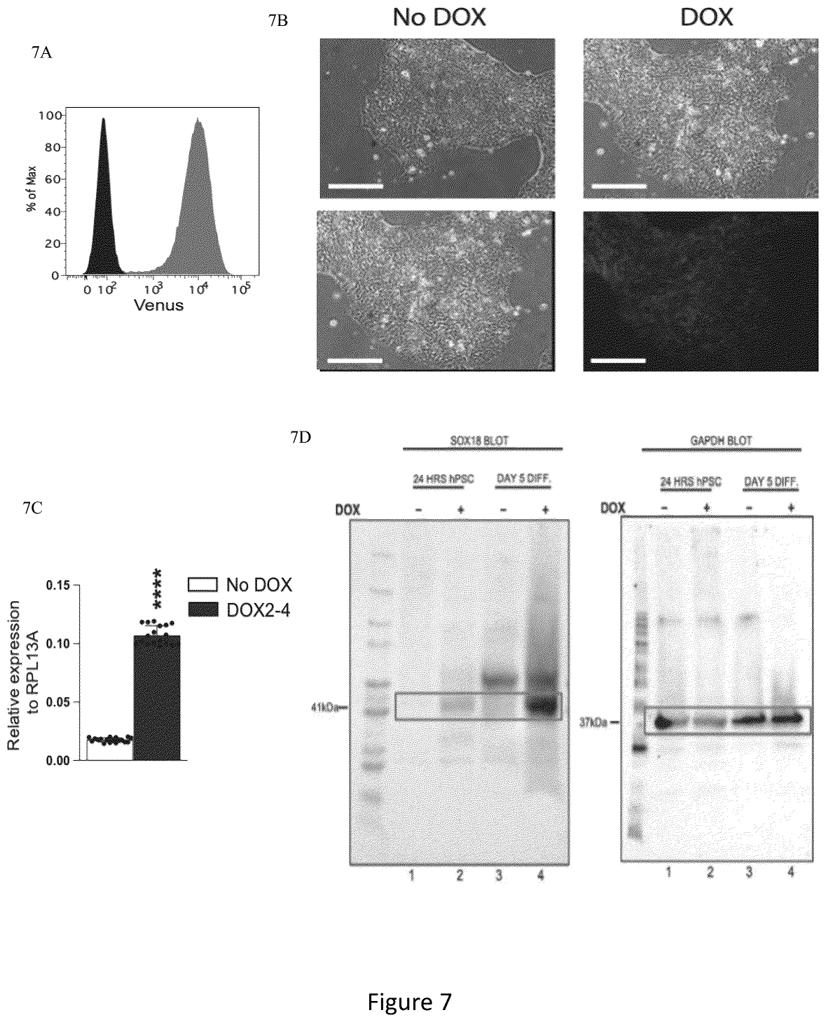

To determine the impact of SOX18 overexpression on hematopoietic development in humans, H1 hESCs carrying doxycycline (DOX)-inducible SOX18-P2A-Venus were generated (FIGS. 1 and 7) and these cells differentiated under a chemically defined culture system in which all stages of hematopoietic development are temporally, phenotypically, and functionally defined (FIG. 2A), as explained with more specificity in Example 2.

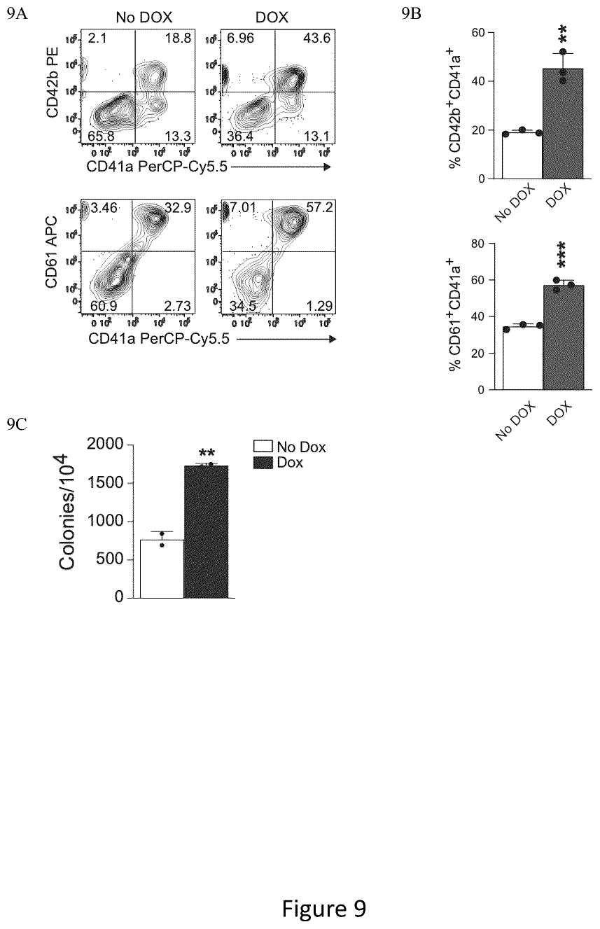

To determine the stages of hematopoietic development sensitive to SOX18 modulation and the optimal duration of SOX18 overexpression to achieve a maximal effect on hematopoietic output, hESC differentiation cultures were treated with DOX at different times and the phenotype and colony-forming cell (CFC) potential of hematopoietic cells collected on day 8 (D8) of differentiation were analyzed (FIG. 2B). As shown in FIGS. 2C-2D, treatment of cultures with DOX from day 2 (D2) through D8 (DOX2-8) resulted in the highest percentages of CD34⁺CD43⁺ HPs and CFCs. This treatment also increased the proportion of CD235a/CD41⁻CD45⁻ progenitors (D8 P1 population) within the CD33⁺CD43⁺ cell population and decreased a proportion of CD235a/CD41a⁺CD45⁺ progenitors (D8 P3 population). Although a similar effect on subset composition within CD34+CD43⁺ progenitors was observed in DOX6-8 and to a lesser degree in DOX4-6 cultures, DOX treatments at these stages of differentiation had a minimal effect on the percentages of CD34+CD43+ cells therein. In contrast, earlier and shorter treatments (DOX2-4) mildly increased CD34+CD43+ cells but had a minimal effect on their composition. These findings suggested that SOX18 has the most profound effect on hematopoietic differentiation when continuously upregulated from D2 through D8 of differentiation. Analysis of colony forming unit (CFC) potential revealed that SOX18 had the most significant effect on GM -and E-CFCs. This was the most pronounced in DOX2-6 and DOX2-8 cultures (FIG. 2E). DOX-treated cultures also had a higher megakaryocytic differentiation potential than non-treated controls (FIG. 9A). These observations indicated that SOX18 overexpression predominantly promoted erythromyeloid progenitors.

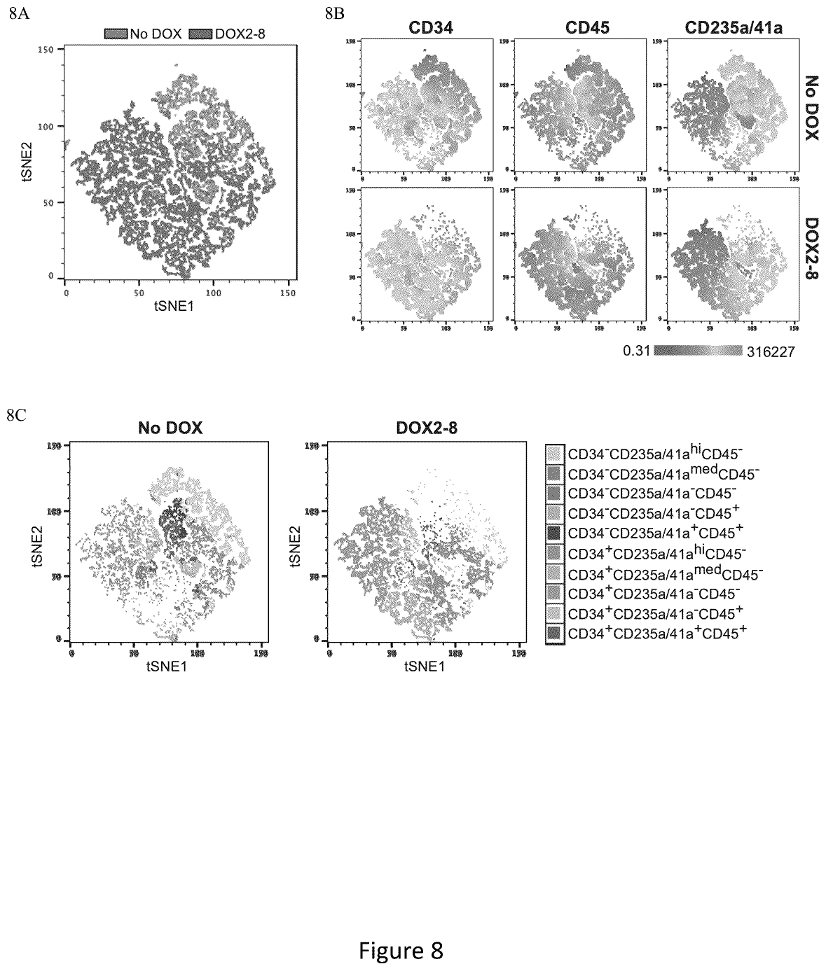

To visualize the in-depth phenotype of D8 hematopoietic cells from No DOX and DOX2-8 cultures, CD43+-gated cells were analyzed using the t-distributed stochastic neighbor embedding algorithm (tSNE) which showed distinct single cell deposition between No DOX and DOX2-8 (FIG. 8A). tSNE analysis also highlighted a suppression of CD45⁺ populations and enrichment of CD45⁻ and CD235a/41a⁻ populations following DOX treatment. In addition, DOX treatment reduced populations with CD235a/41a^{hi/med}CD34⁻ phenotype (FIGS. 8B-8C) corresponding to more mature erythromyeloid progenitors. This observation was consistent with prior findings in murine systems, which demonstrates blocking effect of Sox18 on differentiation of ESC⁻ and yolk sac-derived HPs (see, Serrano et al., 2010, Blood 115: 3895-3898).

### Example 2

### SOX18-Enforced Expression Promotes Megakaryocyte Production

To determine the impact of SOX18 overexpression on hematopoietic development in humans, H1 hESCs carrying doxycycline (DOX)-inducible SOX18-P2A-Venus (FIGS. 1 and 7) were generated and these cells differentiated under a chemically defined culture system in which all stages of hematopoietic development are temporally, phenotypically, and functionally defined. In this differentiation system, the most primitive hemogenic cells with FGF2-dependent hemangioblast colony-forming cell (HB-CFCs) potential are detected on day 3 (D3) of differentiation. The first immature/primordial VEC⁺CD43⁻CD73⁻NOTCH1⁺ HE cells expressing high levels of HAND1 mesodermal gene and lacking arterial and venous gene expression arise on D4 (D4 HE). Subsequently on D5, HE specifies into DLL4⁺CXCR4^{+/-} arterial-type HE with definitive lympho-myeloid potential and DLL4⁻ non-arterial-type HE with myeloid-restricted potential (FIG. 2A). D5 DLL4⁺CXCR4⁺ HE is highly enriched in lymphoid-myeloid progenitors and expresses the highest levels of HOXA and arterial genes, including SOX17 and NOTCH4, as compared to other DLL4⁺ and DLL4⁻ HE populations. All hematopoietic progenitors derived from hPSC cultures on D8 of differentiation can be identified by CD43 expression (Vodyanik et al., Blood 108:2095-2105 (2006); Choi et al., Stem Cell 27:559-567 (2009)) D8 CD34⁺CD43⁺ HPs are composed of at least three major subpopulations: (1) CD235a⁺CD41a⁺CD45⁻ progenitors enriched in erythro-megakaryocytic cells, (2) CD41a^{lo}CD235a^{+/-}CD45⁺ progenitors with erythro-myeloid potential, and (3) CD235a⁻CD41a⁻CD43⁺CD45⁺ multilineage progenitors that are lacking lineage markers, and display CD90⁺CD38⁻CD45RA⁻ phenotype (Vodyanik et al., Blood 108:2095-2105 (2006); Choi et al., Stem Cell 27:559-567 (2009); Suknuntha et al. Stem Cell Res. 15:678-693 (2015); Mesquitta et al. Sci. Rep. 9:6622 (2019)) typical for human hematopoietic stem/progenitor cells (HPSCs) Ivanovs et al., Stem Cell Rep. 2:449-456 (2014). In addition, it has been shown that SOX18 expression is initiated on D4 differentiation in HE and remains present in D5 HE and D8 HPs.

To determine the stages of hematopoietic development sensitive to SOX18 modulation and the optimal duration of SOX18 overexpression to achieve a maximal effect on hematopoietic output, hESC differentiation cultures were treated with DOX at different time points and the phenotype and CFC potential of hematopoietic cells collected on D8 of differentiation analyzed (FIG. 2B). As shown in FIGS. 2C-2D, treatment of cultures with DOX from D2 through D8 (DOX2-8) resulted in the highest percentages of CD34⁺CD43⁺ HPs and CFCs. This treatment increased proportion of CD235a/CD41a⁻CD45⁻ progenitors (D8 P1 population) within CD34⁺CD43⁺ population and decreased a proportion of CD235a/CD41a⁺CD45⁺ progenitors (D8 P3 population). Although a similar effect on subset composition within CD34⁺CD43⁺ progenitors was observed in DOX6-8 and to a lesser degree in DOX4-6 cultures, DOX treatments at these stages of differentiation had a minimal effect on the percentages of CD34⁺CD43⁺ cells. In contrast, earlier and shorter treatments (DOX2-4) mildly increased CD34⁺CD43⁺ cells but had a minimal effect on their composition. These findings suggested that SOX18 had the most profound effect on CD34⁺CD43⁺ HPs and CFCs when continuously upregulated from D2 through D8 of differentiation.

Analysis of CFC potential revealed that SOX18 has the most significant effect on GM- and E-CFCs. This was the most pronounced in DOX2-6 and DOX2-8 cultures (FIG. 2E). Furthermore, DOX treated cultures were found to have a higher megakaryocytic differentiation potential than non-treated controls FIGS. 9A-9B). These observations indicated that SOX18 overexpression starting from D2 of differentiation predominantly promoted erythromyeloid progenitors.

### Example 3

### SOX18-Enforced Expression Suppresses T Cell Differentiation and prOmotes NK Cell Production

To evaluate the effect of SOX18 on lymphoid differentiation, cultures were treated with DOX as shown in FIG. 2B and T cell and NK cell potential of HPs generated on day 8 of 9 differentiation were evaluated. Treatment of differentiation cultures with DOX suppressed T cell potential of day 8 HPs. This suppression of T cell potential was more pronounced in cultures treated with DOX from D2 through D8 (FIG. 2F-2G). However, HP from DOX-treated cultures were able to produce CD56⁺ NK cells, of which the output increased most dramatically following prolonged SOX18 upregulation during hematopoietic differentiation (DOX2-6 and DOX2-8 cultures; FIGS. 2H-2I). These DOX treatments also increased expression of CD16 by CD56⁺ cells, while CD94 expression increased during D2-4 or D2-6 but decreased during D2-8 DOX treatment. Overall these findings suggested that SOX18-enforced expression during day 2-8 (D2-8) of differentiation promotes NK cell development and slightly affects the expression of CD16 and CD94.

### Example 4

### SOX18-Enforced Expression Promotes HB-CFCs but Have a Limited Effect on Specification of Hemogenic Endothelium Formation

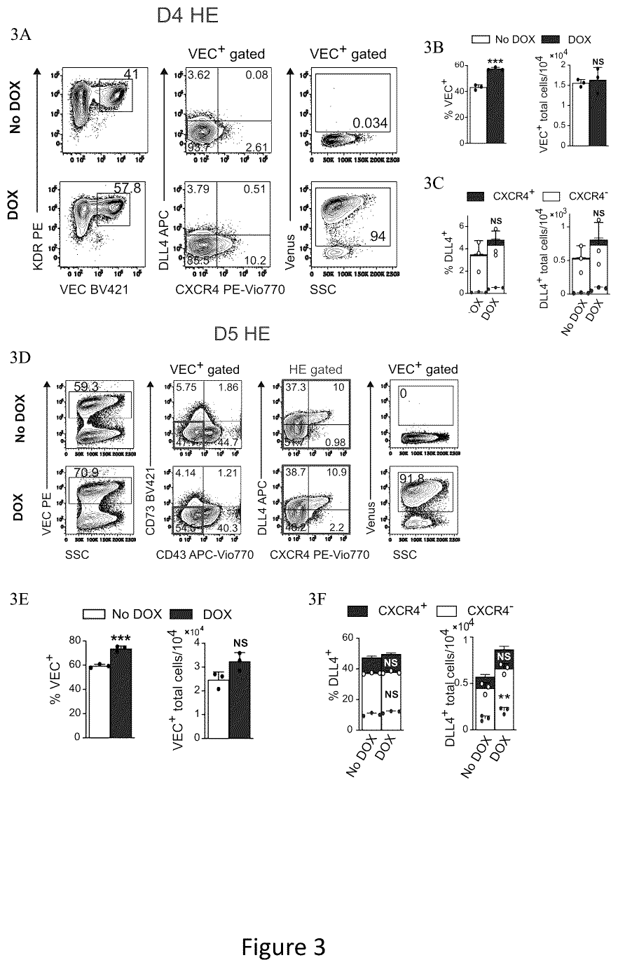

To determine stages of hematopoiesis mostly affected by SOX18 overexpression, the effect of DOX treatment on the formation of the hemangioblast (HB) colonies and HE, including arterial HE specification were analyzed. Enforced expression of SOX18 on D2 of differentiation resulted in almost 3-fold increase in the numbers of HB colonies (FIG. 9B). However, SOX18 overexpression had a limited effect on HE formation and arterial HE specification on D4 and D5 of differentiation (FIGS. 3A-3F). Although a slight increase in the proportion of VE-cadherin⁺ (VEC⁺) endothelial cells in DOX-treated cultures was observed, no significant differences were observed in the proportion of DLL4⁺CXCR4^{+/-} arterial HE in DOX and No DOX cultures. Formation of a DLL4⁻CXCR4⁺ population in DOX-treated D4 cultures was also observed. However, transcriptional profiling of DLL4⁻CXCR4⁺ and DLL4⁻CXCR4⁻D4 VEC⁺ populations revealed no differentially expressed genes (as set forth below).

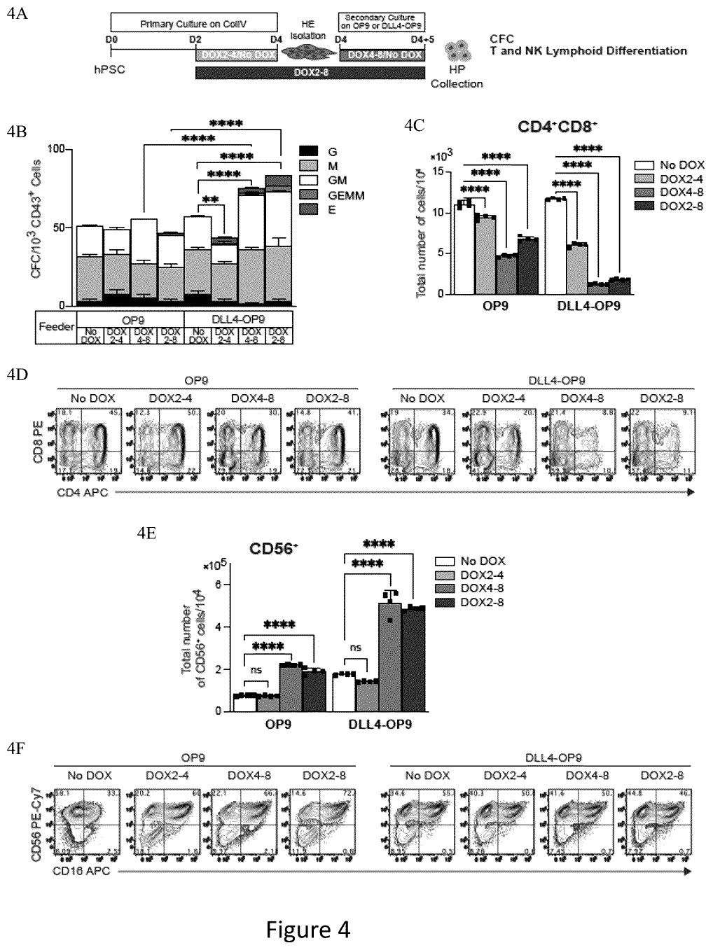

To assess the effect of SOX18 on HE, D4 HE generated in No DOX and DOX conditions were isolated and cultured on OP9 or OP9-DLL4 in presence or absence of DOX (FIG. 4A). Analysis of CFC potential DOX treatment had the most profound effect on HE cultured on OP9-DLL4. In these cocultures, continued treatment with DOX significantly increased CFC formation, while HE from primary differentiation cultures pretreated with DOX (DOX2-4) without additional DOX treatment during cocultures generated fewer CFCs as compared to No DOX controls (FIG. 4B).

Analysis of T cell potential revealed that HE from DOX2-4 treated cultures demonstrated decreased total T cell output when cultured on OP9 and OP9-DLL4. This effect was more pronounced when DOX treatment was initiated on D4 in coculture of HE with OP9 or OP9⁻ DLL4, or in DOX2-8 treatment cultures (FIGS. 4C-4D). In contrast, treatment of HE in OP9 or OP9-DLL4 cultures markedly increased NK cell potential of D8 HPs, while D2-4 DOX treatment of primary differentiation cultures had no effect on NK cells (FIGS. 4E-4F). These findings suggested that SOX18 overexpression predominantly affected lymphoid specification from HE by shifting the balance of NK versus T lymphocyte differentiation potential.

### Example 5

### SOX18-Enforced Expression Promotes Expansion of CD34+CD43+CD235a/CD41a−CD45− (D8 P1) Population Enriched in NK cell Potential

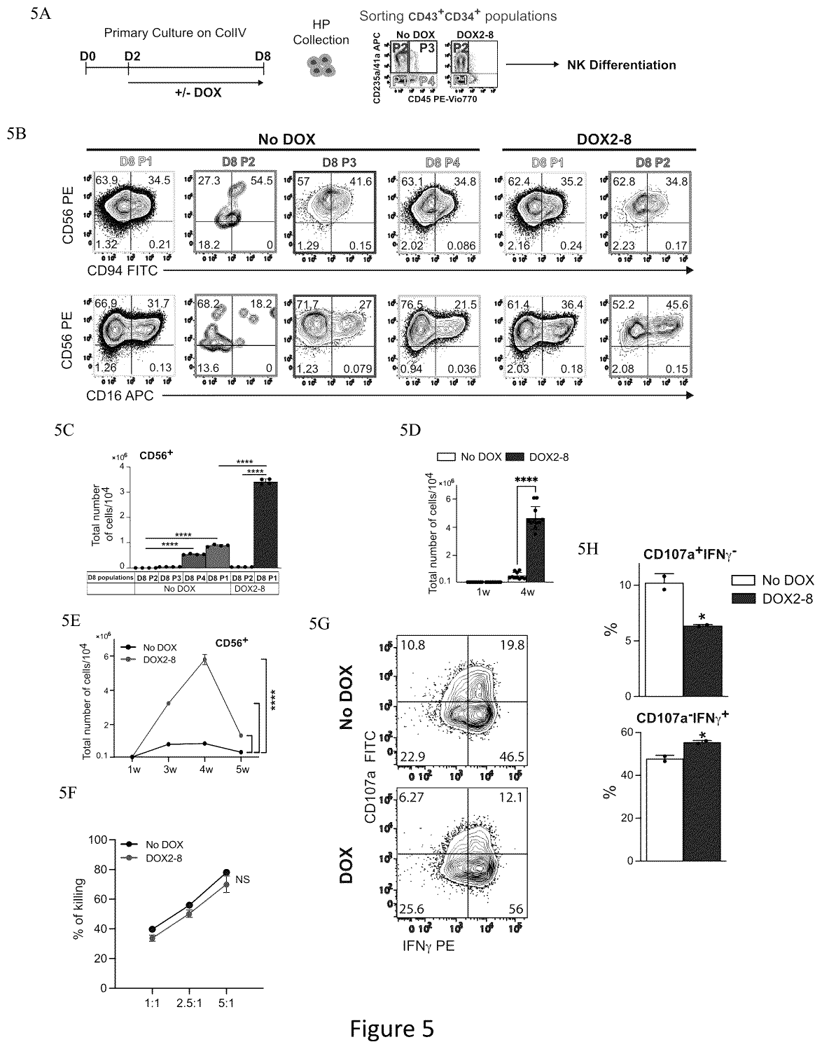

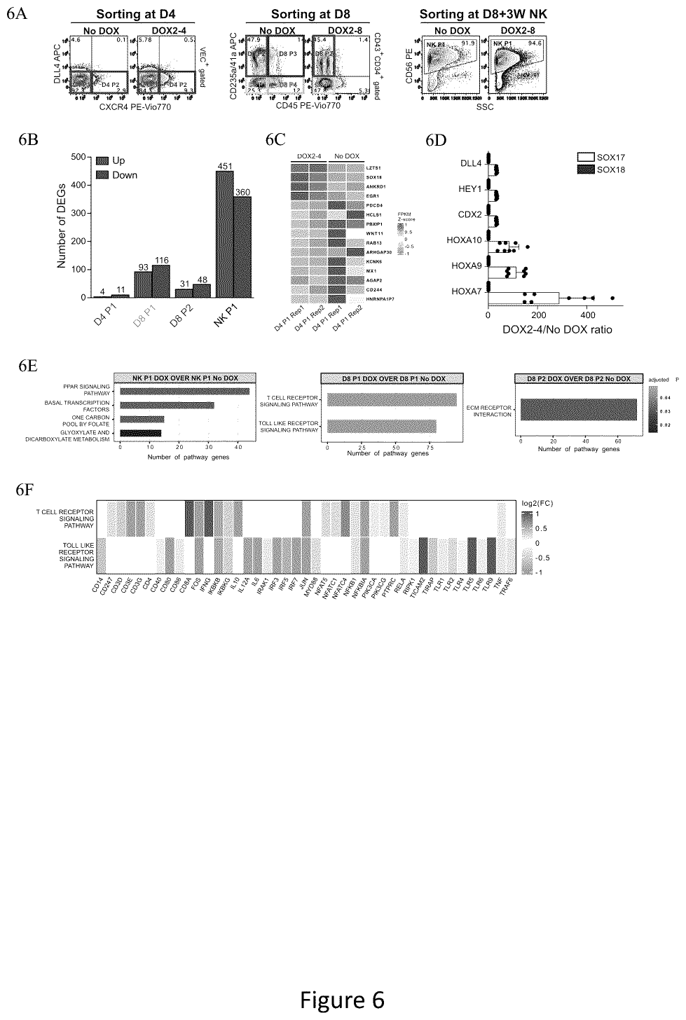

As shown in FIGS. 2C and 2D, D2-D8 SOX18 overexpression significantly promoted development of CD235a/CD41a⁻CD45⁻ (D8 P1) populations and inhibited development of CD45⁺populations, including CD235A/CD41a⁺ (D8 P3) and CD235a/CD41a⁻ (D8 P4) populations within CD34⁺CD43⁺ HPs. To define cell populations enriched in NK cells following DOX treatment, major populations of HPs formed on D8 of culture were isolated and their NK cell potential assessed (FIG. 6A). As shown in FIG. 6B and 6C, in control cultures NK cell potential was mostly detected in D8 P1 and P4 populations, while P2 population failed to produce NK cells. Although CD235a/CD41a⁺CD45⁺ (D8 P3) cells were capable of producing NK cells, the total NK cell output from this subset was negligible, as compared to the P1 and P4 populations. Due to dramatic inhibition of CD45⁺cell development in DOX treated cultures, the NK cell potential of only two major populations were evaluated: i) the D8 P1 population which markedly expanded following DOX treatment and ii) the D8 P2 population. These studies revealed that, in DOX treated cultures, NK cell potential was restricted to the D8 P1 population and that this population possessed much stronger NK cell differentiation potential as compared to the NK-producing populations in the control No DOX cultures (FIG. 5B and 5C). Overall, HPs collected from DOX2-8 treated cultures expanded more rapidly and longer in NK cell differentiation cultures and were able to generate up to 5-fold more NK cells as compared to the control (FIG. 5D). Although differences in cytotoxic potential against K562 cells of NK cells generated from DOX+ and DOX− conditions were not observed (FIG. 5E), in response to PMA, NK cells from DOX-treated cultures exhibited robust IFNγ production, but slightly decreased degranulation response as compared to NK cells from non-treated cultures (FIG. 5F and 5G). Overall, these studies indicated that enforced SOX18 expression predominantly expanded an CD34⁺CD43⁺CD235a⁻/CD41a⁻CD45⁻ (D8 P1) HP population with superior NK cell potential.

### Example 6

### Molecular Characterization of SOX18-Induced Changes in HE and Blood Cells

To define changes in transcriptional program induced by enforced SOX18 expression, RNAseq analysis was performed on D4 HE, D8 CD34+CD43+ subsets and NK cells generated from No DOX and DOX-treated cultures (FIG. 6A). Only 15 differentially expressed genes (DEGs) were detected in the D4 DLL4−CXCR4−VEC+ population (D4 P1 population) following D2-DOX treatment (FIG. 6B), suggesting minimal effect of SOX18 on D4 HE. In contrast to prior findings of D2-4 SOX17 overexpression (see, Jung et al., 2021, Cell Rep. 34: 108758), RNAseq analysis of SOX18 D4 HE from No DOX and DOX cultures showed no significant increase in expression of HOXA genes, CDX2, or genes involved in NOTCH signaling pathways (FIG. 6C). These findings were confirmed using qPCR analysis of selected genes in D4 HE cells isolated from control and D2-4 DOX-treated differentiation cultures of iSOX17 and iSOX18 hPSCs (FIG. 6D). Since flow cytometric analysis showed that SOX18 overexpression induced DLL4−CXCR4+ subset within D4 HE (FIG. 3A and 6A), DEGs were analyzed in DLL4−CXCR4+ (D4 P1) and DLL4−CXCR4− (D4 P2) populations from DOX-treated cultures. These studies revealed no DEGs in these subsets including expression of arterial genes, thus suggesting that CXCR4 expression in D4 HE without co-expression of DLL4 does not reflect activation of arterial program. The most significant changes in transcriptional program were observed in the D8 P1 multipotent HP population and in CD56+NK cells which showed 209 and 811 DEGs in their corresponding DOX treated and untreated cultures (FIG. 6B). Gene set enrichment analysis (GSEA) in D8 P1 cells revealed enrichment in Kyoto Encyclopedia of Genes and Genomes (KEGG) categories related to T cell receptor (TCR) and Toll like receptor (TLR) signaling pathways (FIG. 6E), suggesting that SOX18 overexpression has a major effect on establishing T lymphoid transcriptional program in the D8 P1 multipotent HPs arising from HE. As shown in FIG. 6F, downregulated genes in these categories included CD3 complex genes, CD4, API complex genes JUN and FOS, PI3 signaling genes and the majority of TLR genes, while upregulated genes included IFNG, TICAM2, TLRS and TLR9. D8 P2 population in DOX+ and DOX− conditions showed only 79 DEGs enriched in extracellular matrix (ECM) receptor interactions KEGG categories (FIG. 6E). GSEA in NK cells generated from DOX treated and untreated cultures demonstrated enrichment in KEGG categories related to glyoxylate and dicarboxylate metabolism, one carbon pool by folate, and PPAR signaling pathway (FIG. 6E). However, no differential expression of NK cell activating receptors KLRK1, NCR2, NCR1 or key NK cell transcription factors EOMES and TBX21 was observed between NK cells from DOX− treated and untreated cultures (Data S1). These findings indicated that overexpression of SOX18 during endothelial-to-hematopoietic transition (EHT) has a major impact on the transcriptional program regulating metabolism of NK cells.

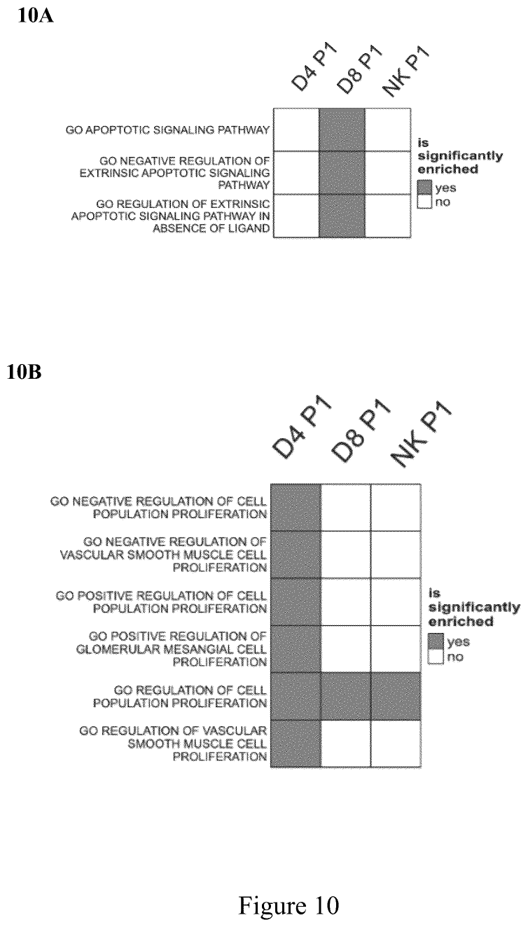

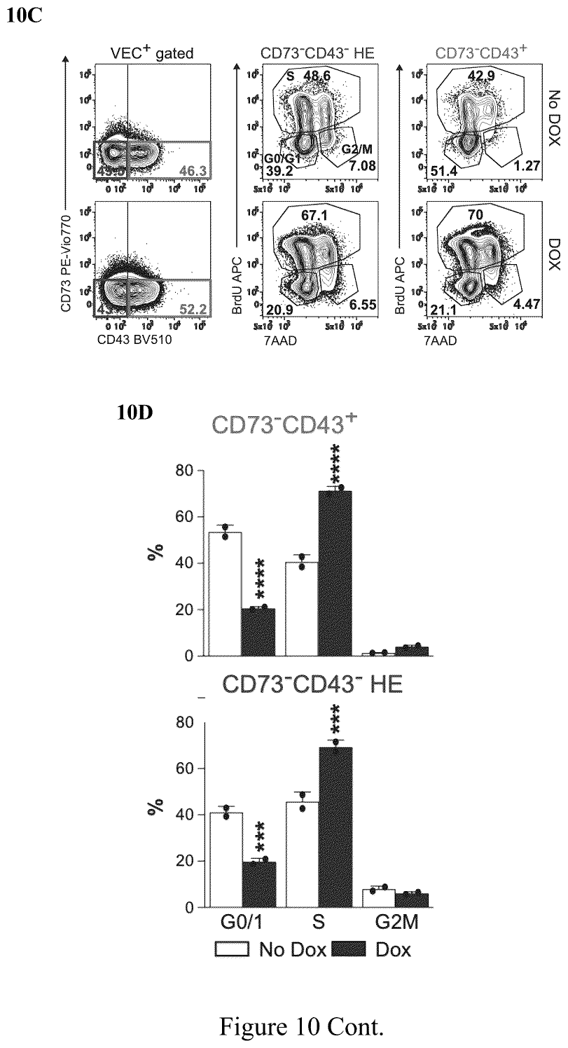

To determine whether SOX18 overexpression affects proliferation and apoptosis in major cell subsets, gene sets enrichment analysis was performed in GO categories related to “Regulation of Cell Population Proliferation” and “Apoptosis”. The first GO gene set was significantly altered in D4 and D8 P1 subsets and NK cells in DOX+ versus No DOX comparisons, whereas apoptotic genes were mostly affected in D8 P1 subset (FIG. 10A and 10B). To confirm the effect of SOX18 on cell proliferation, we performed cell-cycle analysis of D5 differentiated cells using bromodeoxyuridine (BrdU). Consistent with gene expression analysis, SOX18 overexpression led to a significant cell-cycle shift from GO/G1 to S phases in HE cells and emerging CD43+ HPs (FIG. 10C and 10D).

TABLE 1
List of antibodies used in this disclosure

| Antigen | Conjugate | Source | Clone | Cat No. |
|---|---|---|---|---|
| CD4 | APC | BD Biosciences | RPA-T4 | 555349 |
| CD5 | PE-Vio770 | Miltenyi Biotec | REA782 | 130-111-109 |
| CD7 | FITC | BD Biosciences | M-T701 | 555360 |
| CD8 | PE | BD Biosciences | HITBa | 555635 |
| CD18 | APC | BD Biosciences | 3G8 | 561248 |
| CD31 | MicroBeads | Miltenyi Biotec | N/A | 130-091-935 |
| CD34 | PE | BD Biosciences | 581 | 555822 |
| CD41a | APC | BD Biosciences | HIP8 | 559777 |
| CD41a | PerCP-Cy5.5 | BD Biosciences | HIP8 | 340930 |
| CD42b | PE | BD Biosciences | HIP1 | 555473 |
| CD43 | APC-Vio770 | Miltenyi Biotec | DF-T1 | 130-101-174 |
| CD43 | BV421 | BD Biosciences | 1G10 | 562916 |
| CD45 | PE-Vio770 | Miltenyi Biotec | 581 | 130-113-119 |
| CD56 | PE | BD Biosciences | B159 | 555516 |
| CD56 | PE-cy7 | BD Biosciences | B159 | 560916 |
| CD61 | APC | BD Biosciences | VI-PL2 | 564174 |
| CD73 | BV421 | BD Biosciences | AD2 | 562430 |
| CD94 | FITC | BD Biosciences | HP-3D9 | 555888 |
| CD144 | BV421 | BD Biosciences | 55-7H1 | 565670 |
| CD144 | PE | Miltenyi Biotec | REA199 | 130-118-495 |
| CD184 | PE-Vio770 | Miltenyi Biotec | REA649 | 130-120-797 |
| CD235a | APC | BD Biosciences | GA-R2 | 551336 |
| | | | (HIR2) | |
| CD309 | PE | BD Biosciences | 89106 | 560494 |
| DLL4 | APC | Miltenyi Biotec | MHD4-46 | 130-096-560 |
| Ghost | Violet | TONBO | | 13-0879-T100 |
| dye | 540 | Biosciences | | |

TABLE 2
List of Primer Pairs used for RT-qPCR

| SEQ | | Forward | SEQ | Reverse | |
|---|---|---|---|---|---|
| ID | | Sequence | ID | Sequence | |
| NO. | Gene | (5'-3') | NO. | (5'-3') | Purpose |
| 1 | CDX2 | CCGAACAGGG | 2 | AGGTTGGCTC | qPCR |
| | | ACTTGTTTA | | TGGCATTTAT | |
| | | | | A | |
| | | GAG | | G | |
| 3 | DLL4 | CAGTGGGCAG | 4 | ACAGGCAGTG | qPCR |
| | | CGAAGCTAC | | GTAGCCATCC | |
| | | A | | TC | |
| 5 | HEY1 | GGACTATCGG | 6 | TGGGAAGCGT | qPCR |
| | | AGTTTGGGA | | AGTTGTTGAG | |
| | | TTT | | | |
| 7 | HOXA7 | AGGTCCAGGA | 8 | CCAGAGAAGG | qPCR |
| | | TCAGGGTAT | | AGGGATTGAT | |
| | | T | | TC | |
| 9 | HOXA9 | GCGCCTTCTC | 10 | CAGTTCCAGG | qPCR |
| | | TGAAAACAAT | | GTCTGGTGTT | |
| 11 | HOXA10 | GCAAAGAGTG | 12 | CGCTCTCGAG | qPCR |
| | | GTCGGAAGA | | TAAGGTACAT | |
| | | A | | ATTG | |
| 13 | RPL13A | CCTGGAGGAG | 14 | TTGAGGACCT | qPCR |
| | | AAGAGGAAA | | CTGTGTATTT | |
| | | | | G | |
| | | GAGA | | TCAA | |
| 19 | SOX18 | GGCAAAGCGT | 20 | CGGCCGGTAC | |
| | | GGAAGGA | | TTGTAGTTG | |

22-0629-US-PRO_Sequence-Listing_ST25
1. PiggyBack-SOX18 construct sequences
(SEQ ID NO.: 15)
ACTCTTCCTTTTTCAATATTATTGAAGCATTTATCAGGGTTATTG
TCTCATGAGCGGATACATATTTGAATGTATTTAGAAAAATAAACA
AATAGGGGTTCCGCGCACATTTCCCCGAAAAGTGCCACCTAAATT
GTAAGCGTTAATATTTTGTTAAAATTCGCGTTAAATTTTTGTTAA
ATCAGCTCATTTTTTAACCAATAGGCCGAAATCGGCAAAATCCCT
TATAAATCAAAAGAATAGACCGAGATAGGGTTGAGTGTTGTTCCA
GTTTGGAACAAGAGTCCACTATTAAAGAACGTGGACTCCAACGTC
AAAGGGCGAAAAACCGTCTATCAGGGCGATGGCCCACTACGTGAA
CCATCACCCTAATCAAGTTTTTTGGGGTCGAGGTGCCGTAAAGCA
CTAAATCGGAACCCTAAAGGGAGCCCCCGATTTAGAGCTTGACGG
GGAAAGCCGGCGAACGTGGCGAGAAAGGAAGGGAAGAAAGCGAAA
GGAGCGGGCGCTAGGGCGCTGGCAAGTGTAGCGGTCACGCTGCGC
GTAACCACCACACCCGCCGCGCTTAATGCGCCGCTACAGGGCGCG
TCCCATTCGCCATTCAGGCTGCGCAACTGTTGGGAAGGGCGATCG
GTGCGGGCCTCTTCGCTATTACGCCAGCTGGCGAAAGGGGGATGT
GCTGCAAGGCGATTAAGTTGGGTAACGCCAGGGTTTTCCCAGTCA
CGACGTTGTAAAACGACGGCCAGTGAGCGCGCCTCGTTCATTCAC
GTTTTTGAACCCGTGGAGGACGGGCAGACTCGCGGTGCAAATGTG
TTTTACAGCGTGATGGAGCAGATGAAGATGCTCGACACGCTGCAG
AACACGCAGCTAGATTAACCCTAGAAAGATAATCATATTGTGACG
TACGTTAAAGATAATCATGCGTAAAATTGACGCATGTGTTTTATC
GGTCTGTATATCGAGGTTTATTTATTAATTTGAATAGATATTAAG
TTTTATTATATTTACACTTACATACTAATAATAAATTCAACAAAC
AATTTATTTATGTTTATTTATTTATTAAAAAAAAACAAAAACTCA
AAATTTCTTCTATAAAGTAACAAAACTTTTATCGAATTCCTGCAG
CCCGGGGGATCCACTAGTTCTAGAGGGACAGCCCCCCCCCAAAGC
CCCCAGGGATGTAATTACGTCCCTCCCCCGCTAGGGGGCAGCAGC
GAGCCGCCCGGGGCTCCGCTCCGGTCCGGCGCTCCCCCCGCATCC
CCGAGCCGGCAGCGTGCGGGGACAGCCCGGGCACGGGGAAGGTGG
CACGGGATCGCTTTCCTCTGAACGCTTCTCGCTGCTCTTTGAGCC
TGCAGACACCTGGGGGGATACGGGGAAAAGGCCTCCAAGGCCAGC
TTCCCACAATAAGTTGGGTGAATTTTGGCTCATTCCTCCTTTCTA
TAGGATTGAGGTCAGAGCTTTGTGATGGGAATTCTGTGGAATGTG
TGTCAGTTAGGGTGTGGAAAGTCCCGCGATCGATCCTAGCTTCGT
GAGGCTCCGGTGCCCGTCAGTGGGCAGAGCGCACATCGCCCACAG
TCCCCGAGAAGTTGGGGGGAGGGGTCGGCAATTGAACCGGTGCCT
AGAGAAGGTGGCGCGGGGTAAACTGGGAAAGTGATGTCGTGTACT
GGCTCCGCCTTTTTCCCGAGGGTGGGGGAGAACCGTATATAAGTG
CAGTAGTCGCCGTGAACGTTCTTTTTCGCAACGGGTTTGCCGCCA
GAACACAGGTAAGTGCCGTGTGTGGTTCCCGCGGGCCTGGCCTCT
TTACGGGTTATGGCCCTTGCGTGCCTTGAATTACTTCCACCTGGC
TCCAGTACGTGATTCTTGATCCCGAGCTGGAGCCAGGGGGGGGCC
TTGCGCTTTAGGAGCCCCTTCGCCTCGTGCTTGAGTTGAGGCCTG
GCCTGGGCGCTGGGGCCGCCGCGTGCGAATCTGGTGGCACCTTCG
CGCCTGTCTCGCTGCTTTCGATAAGTCTCTAGCCATTTAAAATTT
TTGATGACCTGCTGCGACGCTTTTTTTCTGGCAAGATAGTCTTGT
AAATGCGGGCCAGGATCTGCACACTGGTATTTCGGTTTTTGGGCC
CGCGGCCGGCGACGGGGCCCGTGCGTCCCAGCGCACATGTTCGGC
GAGGCGGGGCCTGCGAGCGCGGCCACCGAGAATCGGACGGGGGTA
GTCTCAAGCTGGCCGGCCTGCTCTGGTGCCTGGCCTCGCGCCGCC
GTGTATCGCCCCGCCCTGGGCGGCAAGGCTGGCCCGGTCGGCACC
AGTTGCGTGAGCGGAAAGATGGCCGCTTCCCGGCCCTGCTCCAGG
GGGCTCAAAATGGAGGACGCGGCGCTCGGGAGAGCGGGCGGGTGA
GTCACCCACACAAAGGAAAAGGGCCTTTCCGTCCTCAGCCGTCGC
TTCATGTGACTCCACGGAGTACCGGGCGCCGTCCAGGCACCTCGA
TTAGTTCTGGAGCTTTTGGAGTACGTCGTCTTTAGGTTGGGGGGA
GGGGTTTTATGCGATGGAGTTTCCCCACACTGAGTGGGTGGAGAC
TGAAGTTAGGCCAGCTTGGCACTTGATGTAATTCTCGTTGGAATT
TGCCCTTTTTGAGTTTGGATCTTGGTTCATTCTCAAGCCTCAGAC
AGTGGTTCAAAGTTTTTTTCTTCCATTTCAGGTGTCGTGAACACG
TGGTCGCGGCCATCTAGAGCAACTAGCATGGCCAAGTTGACCAGT
GCCGTTCCGGTGCTCACCGCGCGCGACGTCGCCGGAGCGGTCGAG
TTCTGGACCGACCGGCTCGGGTTCTCCCGGGACTTCGTGGAGGAC
GACTTCGCCGGTGTGGTCCGGGACGACGTGACCCTGTTCATCAGC
GCGGTCCAGGACCAGGTGGTGCCGGACAACACCCTGGCCTGGGTG
TGGGTGCGCGGCCTGGACGAGCTGTACGCCGAGTGGTCGGAGGTC
GTGTCCACGAACTTCCGGGACGCCTCCGGGCCGGCCATGACCGAG
ATCGGCGAGCAGCCGTGGGGGGGGAGTTCGCCCTGCGCGACCCGG
CCGGCAACTGCGTGCACTTCGTGGCCGAGGAGCAGGACTGAATCT
AAGATACATTGATGAGTTTGGACAAACCACAACTAGAATGCAGTG
AAAAAAATGCTTTATTTGTGAAATTTGTGATGCTATTGCTTTATT
TGTAACCATTATAAGCTGCAATAAACAAGTTAACAACAACAATTG
CATTCATTTTATGTTTCAGGTTCAGGGGGAGGTGTGGGAGGTTTT
TTAAAGCAAGTAAAACCTCTACAAATGTGGTATGGCTGATTATGA
TCCGGCTGCCTCGCGCATGCATCATAGAGCCCACCGCATCCCCAG
CATGCCTGCTATTGTCTTCCCAATCCTCCCCCTTGCTGTCCTGCC
CCACCCCACCCCCCAGAATAGAATGACACCTACTCAGACAATGCG
ATGCAATTTCCTCATTTTATTAGGAAAGGACAGTGGGAGTGGCAC
CTTCCAGGGTCAAGGAAGGCACGGGGGAGGGGCAAACAACAGATG
GCTGGCAACTAGAAGGCACAGTCGAGGCTGATCAGCCCATGATTA
CTTGTACAGCTCGTCCATGCCGAGAGTGATCCCGGCGGCGGTCAC
GAACTCCAGCAGGACCATGTGATCGCGCTTCTCGTTGGGGTCTTT
GCTCAGGGCGGACTGGTAGCTCAGGTAGTGGTTGTCGGGCAGCAG
CACGGGGCCGTCGCCGATGGGGGTGTTCTGCTGGTAGTGGTCGGC
GAGCTGCACGCCGCCGTCCTCGATGTTGTGGCGGATCTTGAAGTT
GGCCTTGATGCCGTTCTTCTGCTTGTCGGCGGTGATATAGACGTT
GTGGCTGTTGTAGTTGTACTCCAGCTTGTGCCCCAGGATGTTGCC
GTCCTCCTTGAAGTCGATGCCCTTCAGCTCGATGCGGTTCACCAG
GGTGTCGCCCTCGAACTTCACCTCGGCGCGGGTCTTGTAGTTGCC
GTCGTCCTTGAAGAAGATGGTGCGCTCCTGGACGTAGCCTTCGGG
CATGGCGGACTTGAAGAAGTCGTGCTGCTTCATGTGGTCGGGGTA
GCGGGCGAAGCACTGCAGGCCGTAGCCCAGGGTGGTCACGAGGGT
GGGCCAGGGCACGGGCAGCTTGCCGGTGGTGCAGATCAGCTTCAG
GGTCAGCTTGCCGTAGGTGGCATCGCCCTCGCCCTCGCCGGACAC
GCTGAACTTGTGGCCGTTTACGTCGCCGTCCAGCTCGACCAGGAT
GGGCACCACCCCGGTGAACAGCTCCTCGCCCTTGCTCACCATGGG
TCCAGGGTTCTCCTCCACGTCTCCAGCCTGCTTCAGCAGGCTGAA
GTTAGTAGCTCCGCTTCCGTATACGCCGGAGATGCACGCGCTGTA
ATAGACCGCGCTGCTGGCGTCCGACAGCGCGGAGATCAGGCTGCT
CTCCTCTGGGCAGGACATGGCGCGCGGGCCCAGTTTGGCCAGTGC
CACGTGGTACGGGAGCCCGGGGGCGTCGGGCCGAGTCCGGCTGCA
GTTGAGGTACTGGTCGAACTCGGTGAGGTCCACGTCGGCCCACAG
ATCGGCGGCGGGCCCCAGCGGCTCGGCGCTCTCCAGCGGCGGGGC
CTCGGGCGGCGGCGACAGCGGGCCGGGGTACGGGCCGGGCGTGCC
CAGGGTGCCGTAGTACAGGCCAGCGAGCGGCGCCGCGGGGGGCGC
GGTCCTGAGCGCCTCCGCCAGGGGAGCCCCGTAGCAACCGCCGGG
GTCCCGCGACAACTCGGTGGGCGCGTAGGGCGCGCGGAAGGGCCG
CAGCGCGCAGTCCTCGGGCGCCGCGGGCGGTGGGAAGAAGGCAGC
CTCGCCGGGCTCCAGGCCGTCCAGAGGCGAGCGCTCGGGCGTGGG
CAGCCCCAGGCCGTCGAACTCGGCGCCCAGCGGGGGCAGCTCGCG
GAAGGCGCGAGCCGAGCCAGACGCCGCGGGGAAAGGCTCGGGCGG
TGGCTGCGGGGGCGCTAATCCCGGGAGCAGGAGGCCGGGCTCCAG
CCGCCGGGCCTTGCGCGCCTGCTTCTTGCGGCGCGGCCGGTACTT
GTAGTTGGGGTGGTCGCGCAAGTGCTGCACGCGCAGCCGTTCGGC
TTCCTCCACGAAGGGCCGCTTCTCCGCCGCGTTCAGCTCCTTCCA
CGCTTTGCCCAGCATCTTGCTGAGCACCGCGTTGTGCAGGTCCGG
GTTCTGCTGAGCCAGCCGCTTGCGCTCGTCCTTTGCCCACACCAT
GAAGGCGTTCATGGGCCGCCGGATGCGCGACTCGTCTGCCGCCTG
GCGTTCCCCGCGGCCGGCCGGGCTGAGGCCATAGCGCCCCGGCTC
GGGGCTGCGCGGGGGACTGCGCTGCGGGCTGGGCGGCGAGGCGGG
CGCGGCGGGCGCGGCGAGGGCGGCGGGGCCGGCGGCGAGGCCGCG
CGTGTCAGCGGCGGCCCCGTGTCCCGGGGCCCATGCACAGTCGCG
GCGGGCGGGCGGGTCGTCCTGTGCGCCGTAGCCGGGCGGCGATCT
CTGCATGGATCCTCCAGGCGATCTGACGGTTCACTAAACGAGCTC
TGCTTATATAGGCCTCCCACCGTACACGCCTACCTCGACATACGT
TCTCTATCACTGATAGGGAGTAAACTCGACATACGTTCTCTATCA
CTGATAGGGATAAACTCGACATACGTTCTCTATCACTGATAGGGA
GTAAACTCGACATACGTTCTCTATCACTGATAGGGAGTAAACTCG
ACATACGTTCTCTATCACTGATAGGGAGTAAACTCGACATCGTTC
TCTATCACTGATAGGGAGTAAACTCGACATACGTTCTCTATCACT
GATAGGGAGTAAACTCGCTCGAGACGCGTATACCTAGGATCCGGC
CGGCCTGCAGGTGTCCTCACAGGAACGAAGTCCCTAAAGAAACAG
TGGCAGCCAGGTTTAGCCCCGGAATTGACTGGATTCCTTTTTTAG
GGCCCATTGGTATGGCTTTTTCCCCGTATCCCCCCAGGTGTCTGC
AGGCTCAAAGAGCAGCGAGAAGCGTTCAGAGGAAAGCGATCCCGT
GCCACCTTCCCCGTGCCCGGGCTGTCCCCGCACGCTGCCGGCTCG
GGGATGCGGGGGGAGCGCCGGACCGGAGCGGAGCCCCGGGCGGCT
CGCTGCTGCCCCCTAGCGGGGGAGGGACGTAATTACATCCCTGGG
GGCTTTGGGGGGGGGCTGTCCCTCTAGAGCGGCCGCCACCGCGGT
GGAGCTCCAGCTTTTGTTCCCTTTAGTGAGGGTTAATTAGATCTT
AATACGACTCACTATAGGGCGAATTGGGTACCGGGCCCCCCCTCG
AGGTCGACGGTATCGATAAGCTTGATATCTATAACAAGAAAATAT
ATATATAATAAGTTATCACGTAAGTAGAACATGAAATAACAATAT
AATTATCGTATGAGTTAAATCTTAAAAGTCACGTAAAAGATAATC
ATGCGTCATTTTGACTCACGCGGTCGTTATAGTTCAAAATCAGTG
ACACTTACCGCATTGACAAGCACGCCTCACGGGAGCTCCAAGCGG
CGACTGAGATGTCCTAAATGCACAGCGACGGATTCGCGCTATTTA
GAAAGAGAGAGCAATATTTCAAGAATGCATGCGTCAATTTTACGC
AGACTATCTTTCTAGGGTTAATCTAGCTGCATCAGGATCATATCG
TCGGGTCTTTTTTCCGGCTCAGTCATCGCCCAAGCTGGCGCTATC
TGGGCATCGGGGAGGAAGAAGCCCGTGCCTTTTCCCGCGAGGTTG
AAGCGGCATGGAAAGAGTTTGCCGAGGATGACTGCTGCTGCATTG
ACGTTGAGCGAAAACGCACGTTTACCATGATGATTCGGGAAGGTG
TGGCCATGCACGCCTTTAACGGTGAACTGTTCGTTCAGGCCACCT
GGGATACCAGTTCGTCGCGGCTTTTCCGGACACAGTTCCGGATGG
TCAGCCCGAAGCGCATCAGCAACCCGAACAATACCGGCGACAGCC
GGAACTGCCGTGCCGGTGTGCAGATTAATGACAGCGGTGCGGCGC
TGGGATATTACGTCAGCGAGGACGGGTATCCTGGCTGGATGCCGC
AGAAATGGACATGGATACCCCGTGAGTTACCCGGCGGGCGCGCTT
GGCGTAATCATGGTCATAGCTGTTTCCTGTGTGAAATTGTTATCC
GCTCACAATTCCACACAACATACGAGCCGGAAGCATAAAGTGTAA
AGCCTGGGGTGCCTAATGAGTGAGCTAACTCACATTAATTGCGTT
GCGCTCACTGCCCGCTTTCCAGTCGGGAAACCTGTCGTGCCAGCT
GCATTAATGAATCGGCCAACGCGCGGGGAGAGGCGGTTTGCGTAT
TGGGCGCTCTTCCGCTTCCTCGCTCACTGACTCGCTGCGCTCGGT
CGTTCGGCTGCGGCGAGCGGTATCAGCTCACTCAAAGGCGGTAAT
ACGGTTATCCACAGAATCAGGGGATAACGCAGGAAAGAACATGTG
AGCAAAAGGCCAGCAAAAGGCCAGGAACCGTAAAAAGGCCGCGTT
GCTGGCGTTTTTCCATAGGCTCCGCCCCCCTGACGAGCATCACAA
AAATCGACGCTCAAGTCAGAGGTGGCGAAACCCGACAGGACTATA
AAGATACCAGGCGTTTCCCCCTGGAAGCTCCCTCGTGCGCTCTCC
TGTTCCGACCCTGCCGCTTACCGGATACCTGTCCGCCTTTCTCCC
TTCGGGAAGCGTGGCGCTTTCTCATAGCTCACGCTGTAGGTATCT
CAGTTCGGTGTAGGTCGTTCGCTCCAAGCTGGGCTGTGTGCACGA
ACCCCCCGTTCAGCCCGACCGCTGCGCCTTATCCGGTAACTATCG
TCTTGAGTCCAACCCGGTAAGACACGACTTATCGCCACTGGCAGC
AGCCACTGGTAACAGGATTAGCAGAGCGAGGTATGTAGGCGGTGC
TACAGAGTTCTTGAAGTGGTGGCCTAACTACGGCTACACTAGAAG
GACAGTATTTGGTATCTGCGCTCTGCTGAAGCCAGTTACCTTCGG
AAAAAGAGTTGGTAGCTCTTGATCCGGCAAACAAACCACCGCTGG
TAGCGGTGGTTTTTTTGTTTGCAAGCAGCAGATTACGCGCAGAAA
AAAAGGATCTCAAGAAGATCCTTTGATCTTTTCTACGGGGTCTGA
CGCTCAGTGGAACGAAAACTCACGTTAAGGGATTTTGGTCATGAG
ATTATCAAAAAGGATCTTCACCTAGATCCTTTTAAATTAAAAATG
AAGTTTTAAATCAATCTAAAGTATATATGAGTAAACTTGGTCTGA
CAGTTACCAATGCTTAATCAGTGAGGCACCTATCTCAGCGATCTG
TCTATTTCGTTCATCCATAGTTGCCTGACTCCCCGTCGTGTAGAT
AACTACGATACGGGAGGGCTTACCATCTGGCCCCAGTGCTGCAAT
GATACCGCGAGACCCACGCTCACCGGCTCCAGATTTATCAGCAAT
AAACCAGCCAGCCGGAAGGGCCGAGCGCAGAAGTGGTCCTGCAAC
TTTATCCGCCTCCATCCAGTCTATTAATTGTTGCCGGGAAGCTAG
AGTAAGTAGTTCGCCAGTTAATAGTTTGCGCAACGTTGTTGCCAT
TGCTACAGGCATCGTGGTGTCACGCTCGTCGTTTGGTATGGCTTC
ATTCAGCTCCGGTTCCCAACGATCAAGGCGAGTTACATGATCCCC
CATGTTGTGCAAAAAAGCGGTTAGCTCCTTCGGTCCTCCGATCGT
TGTCAGAAGTAAGTTGGCCGCAGTGTTATCACTCATGGTTATGGC
AGCACTGCATAATTCTCTTACTGTCATGCCATCCGTAAGATGCTT
TTCTGTGACTGGTGAGTACTCAACCAAGTCATTCTGAGAATAGTG
TATGCGGCGACCGAGTTGCTCTTGCCCGGCGTCAATACGGGATAA
TACCGCGCCACATAGCAGAACTTTAAAAGTGCTCATCATTGGAAA
ACGTTCTTCGGGGCGAAAACTCTCAAGGATCTTACCGCTGTTGAG
ATCCAGTTCGATGTAACCCACTCGTGCACCCAACTGATCTTCAGC
ATCTTTTACTTTCACCAGCGTTTCTGGGTGAGCAAAAACAGGAAG
GCAAAATGCCGCAAAAAAGGGAATAAGGGCGACACGGAAATGTTG
AATACTCAT
1A.
Human SOX18 CDS (ORF)
(SEQ ID NO.: 16)
***
ATGCAGAGATCGCCGCCCGGCTACGGCGCACAGGACGACCCGCCC
GCCCGCCGCGACTGTGCATGGGCCCCGGGACACGGGGCCGCCGCT
GACACGCGCGGCCTCGCCGCCGGCCCCGCCGCCCTCGCCGCGCCC
GCCGCGCCCGCCTCGCCGCCCAGCCCGCAGCGCAGTCCCCCGCGC
AGCCCCGAGCCGGGGCGCTATGGCCTCAGCCCGGCCGGCCGCGGG
GAACGCCAGGCGGCAGACGAGTCGCGCATCCGGCGGCCCATGAAC
GCCTTCATGGTGTGGGCAAAGGACGAGCGCAAGCGGCTGGCTCAG
CAGAACCCGGACCTGCACAACGCGGTGCTCAGCAAGATGCTGGGC
AAAGCGTGGAAGGAGCTGAACGCGGCGGAGAAGCGGCCCTTCGTG
GAGGAAGCCGAACGGCTGCGCGTGCAGCACTTGCGCGACCACCCC
AACTACAAGTACCGGCCGCGCCGCAAGAAGCAGGCGCGCAAGGCC
CGGCGGCTGGAGCCCGGCCTCCTGCTCCCGGGATTAGCGCCCCCG
CAGCCACCGCCCGAGCCTTTCCCCGCGGCGTCTGGCTCGGCTCGC
GCCTTCCGCGAGCTGCCCCCGCTGGGCGCCGAGTTCGACGGCCTG
GGGCTGCCCACGCCCGAGCGCTCGCCTCTGGACGGCCTGGAGCCC
GGCGAGGCTGCCTTCTTCCCACCGCCCGCGGCGCCCGAGGACTGC
GCGCTGCGGCCCTTCCGCGCGCCCTACGCGCCCACCGAGTTGTCG
CGGGACCCCGGCGGTTGCTACGGGGCTCCCCTGGCGGAGGCGCTC
AGGACCGCGCCCCCCGCGGCGCCGCTCGCTGGCCTGTACTACGGC
ACCCTGGGCACGCCCGGCCCGTACCCCGGCCCGCTGTCGCCGCCG
CCCGAGGCCCCGCCGCTGGAGAGCGCCGAGCCGCTGGGGCCCGCC
GCCGATCTGTGGGCCGACGTGGACCTCACCGAGTTCGACCAGTAC
CTCAACTGCAGCCGGACTCGGCCCGACGCCCCCGGGCTCCCGTAC
CACGTGGCACTGGCCAAACTGGGCCCGCGCGCCATGTCCTGCCCA
GAGGAGAGCAGCCTGATCTCCGCGCTGTCGGACGCCAGCAGCGCG
GTCTATTACAGCGCGTGCATCTCCGGC
*** = start (ATG) codon
SOX18 amino acid sequence
(SEQ ID NO.: 17)
MQRSPPGYGAQDDPPARRDCAWAPGHGAAADTRGLAAGPAALAAP
AAPASPPSPQRSPPRSPEPGRYGLSPAGRGERQAADESRIRRPMN
AFMVWAKDERKRLAQQNPDLHNAVLSKMLGKAWKELNAAEKRPFV
EEAERLRVQHLRDHPNYKYRPRRKKQARKARRLEPGLLLPGLAPP
QPPPEPFPAASGSARAFRELPPLGAEFDGLGLPTPERSPLDGLEP
GEAAFFPPPAAPEDCALRPFRAPYAPTELSRDPGGCYGAPLAEAL
RTAPPAAPLAGLYYGTLGTPGPYPGPLSPPPEAPPLESAEPLGPA
ADLWADVDLTEFDQYLNCSRTRPDAPGLPYHVALAKLGPRAMSCP
EESSLISALSDASSAVYYSACISG
2. PB-M2rtTA sequences (regulatory element
for DOX-inducible SOX18 expression)
(SEQ ID NO.: 18)
ACTCTTCCTTTTTCAATATTATTGAAGCATTTATCAGGGTTATTG
TCTCATGAGCGGATACATATTTGAATGTATTTAGAAAAATAAACA
AATAGGGGTTCCGCGCACATTTCCCCGAAAAGTGCCACCTAAATT
GTAAGCGTTAATATTTTGTTAAAATTCGCGTTAAATTTTTGTTAA
ATCAGCTCATTTTTTAACCAATAGGCCGAAATCGGCAAAATCCCT
TATAAATCAAAAGAATAGACCGAGATAGGGTTGAGTGTTGTTCCA
GTTTGGAACAAGAGTCCACTATTAAAGAACGTGGACTCCAACGTC
AAAGGGCGAAAAACCGTCTATCAGGGCGATGGCCCACTACGTGAA
CCATCACCCTAATCAAGTTTTTTGGGGTCGAGGTGCCGTAAAGCA
CTAAATCGGAACCCTAAAGGGAGCCCCCGATTTAGAGCTTGACGG
GGAAAGCCGGCGAACGTGGCGAGAAAGGAAGGGAAGAAAGCGAAA
GGAGCGGGCGCTAGGGCGCTGGCAAGTGTAGCGGTCACGCTGCGC
GTAACCACCACACCCGCCGCGCTTAATGCGCCGCTACAGGGCGCG
TCCCATTCGCCATTCAGGCTGCGCAACTGTTGGGAAGGGCGATCG
GTGCGGGCCTCTTCGCTATTACGCCAGCTGGCGAAAGGGGGATGT
GCTGCAAGGCGATTAAGTTGGGTAACGCCAGGGTTTTCCCAGTCA
CGACGTTGTAAAACGACGGCCAGTGAGCGCGCCTCGTTCATTCAC
GTTTTTGAACCCGTGGAGGACGGGCAGACTCGCGGTGCAAATGTG
TTTTACAGCGTGATGGAGCAGATGAAGATGCTCGACACGCTGCAG
AACACGCAGCTAGATTAACCCTAGAAAGATAATCATATTGTGACG
TACGTTAAAGATAATCATGCGTAAAATTGACGCATGTGTTTTATC
GGTCTGTATATCGAGGTTTATTTATTAATTTGAATAGATATTAAG
TTTTATTATATTTACACTTACATACTAATAATAAATTCAACAAAC
AATTTATTTATGTTTATTTATTTATTAAAAAAAAACAAAAACTCA
AAATTTCTTCTATAAAGTAACAAAACTTTTATCGAATTCCTGCAG
CCCGGGGGATCCACTAGTTCTAGAGGGACAGCCCCCCCCCAAAGC
CCCCAGGGATGTAATTACGTCCCTCCCCCGCTAGGGGGCAGCAGC
GAGCCGCCCGGGGCTCCGCTCCGGTCCGGCGCTCCCCCCGCATCC
CCGAGCCGGCAGCGTGCGGGGACAGCCCGGGCACGGGGAAGGTGG
CACGGGATCGCTTTCCTCTGAACGCTTCTCGCTGCTCTTTGAGCC
TGCAGACACCTGGGGGGATACGGGGAAAAGGCCTCCAAGGCCAGC
TTCCCACAATAAGTTGGGTGAATTTTGGCTCATTCCTCCTTTCTA
TAGGATTGAGGTCAGAGCTTTGTGATGGGAATTCTGTGGAATGTG
TGTCAGTTAGGGTGTGGAAAGTCCCGCGATCGTCGAGAGATTTCT
AGCTTCGTGAGGCTCCGGTGCCCGTCAGTGGGCAGAGCGCACATC
GCCCACAGTCCCCGAGAAGTTGGGGGGAGGGGTCGGCAATTGAAC
CGGTGCCTAGAGAAGGTGGCGCGGGGTAAACTGGGAAAGTGATGT
CGTGTACTGGCTCCGCCTTTTTCCCGAGGGTGGGGGAGAACCGTA
TATAAGTGCAGTAGTCGCCGTGAACGTTCTTTTTCGCAACGGGTT
TGCCGCCAGAACACAGGTAAGTGCCGTGTGTGGTTCCCGCGGGCC
TGGCCTCTTTACGGGTTATGGCCCTTGCGTGCCTTGAATTACTTC
CACCTGGCTCCAGTACGTGATTCTTGATCCCGAGCTGGAGCCAGG
GGGGGGCCTTGCGCTTTAGGAGCCCCTTCGCCTCGTGCTTGAGTT
GAGGCCTGGCCTGGGCGCTGGGGCCGCCGCGTGCGAATCTGGTGG
CACCTTCGCGCCTGTCTCGCTGCTTTCGATAAGTCTCTAGCCATT
TAAAATTTTTGATGACCTGCTGCGACGCTTTTTTTCTGGCAAGAT
AGTCTTGTAAATGCGGGCCAGGATCTGCACACTGGTATTTCGGTT
TTTGGGCCCGCGGCCGGCGACGGGGCCCGTGCGTCCCAGCGCACA
TGTTCGGCGAGGCGGGGCCTGCGAGCGCGGCCACCGAGAATCGGA
CGGGGGTAGTCTCAAGCTGGCCGGCCTGCTCTGGTGCCTGGCCTC
GCGCCGCCGTGTATCGCCCCGCCCTGGGCGGCAAGGCTGGCCCGG
TCGGCACCAGTTGCGTGAGCGGAAAGATGGCCGCTTCCCGGCCCT
GCTCCAGGGGGCTCAAAATGGAGGACGCGGCGCTCGGGAGAGCGG
GCGGGTGAGTCACCCACACAAAGGAAAAGGGCCTTTCCGTCCTCA
GCCGTCGCTTCATGTGACTCCACGGAGTACCGGGCGCCGTCCAGG
CACCTCGATTAGTTCTGGAGCTTTTGGAGTACGTCGTCTTTAGGT
TGGGGGGAGGGGTTTTATGCGATGGAGTTTCCCCACACTGAGTGG
GTGGAGACTGAAGTTAGGCCAGCTTGGCACTTGATGTAATTCTCG
TTGGAATTTGCCCTTTTTGAGTTTGGATCTTGGTTCATTCTCAAG
CCTCAGACAGTGGTTCAAAGTTTTTTTCTTCCATTTCAGGTGTCG
TGAACACGTGGTCGCGGCCATCTAGAGCAACTAGCTTGGGCCCGG
GAATTAATTCACCATGTCTAGACTGGACAAGAGCAAAGTCATAAA
CGGCGCTCTGGAATTACTCAATGGAGTCGGTATCGAAGGCCTGAC
GACAAGGAAACTCGCTCAAAAGCTGGGAGTTGAGCAGCCTACCCT
GTACTGGCACGTGAAGAACAAGCGGGCCCTGCTCGATGCCCTGCC
AATCGAGATGCTGGACAGGCATCATACCCACTTCTGCCCCCTGGA
AGGCGAGTCATGGCAAGACTTTCTGCGGAACAACGCCAAGTCATT
CCGCTGTGCTCTCCTCTCACATCGCGACGGGGCTAAAGTGCATCT
CGGCACCCGCCCAACAGAGAAACAGTACGAAACCCTGGAAAATCA
GCTCGCGTTCCTGTGTCAGCAAGGCTTCTCCCTGGAGAACGCACT
GTACGCTCTGTCCGCCGTGGGCCACTTTACACTGGGCTGCGTATT
GGAGGAACAGGAGCATCAAGTAGCAAAAGAGGAAAGAGAGACACC
TACCACCGATTCTATGCCCCCACTTCTGAGACAAGCAATTGAGCT
GTTCGACCGGCAGGGAGCCGAACCTGCCTTCCTTTTCGGCCTGGA
ACTAATCATATGTGGCCTGGAGAAACAGCTAAAGTGCGAAAGCGG
CGGGCCGGCCGACGCCCTTGACGATTTTGACTTAGACATGCTCCC
AGCCGATGCCCTTGACGACTTTGACCTTGATATGCTGCCTGCTGA
CGCTCTTGACGATTTTGACCTTGACATGCTCCCCGGGGAATTCGG
AAGCGGAGAGGGCAGAGGAAGTCTTCTAACATGCGGTGACGTGGA
GGAGAATCCCGGCCCTGATATCATGACCGAGTACAAGCCCACGGT
GCGCCTCGCCACCCGCGACGACGTCCCCCGGGCCGTACGCACCCT
CGCCGCCGCGTTCGCCGACTACCCCGCCACGCGCCACACCGTCGA
CCCGGACCGCCACATCGAGCGGGTCACCGAGCTGCAAGAACTCTT
CCTCACGCGCGTCGGGCTCGACATCGGCAAGGTGTGGGTCGCGGA
CGACGGCGCCGCGGTGGCGGTCTGGACCACGCCGGAGAGCGTCGA
AGCGGGGGCGGTGTTCGCCGAGATCGGCCCGCGCATGGCCGAGTT
GAGCGGTTCCCGGCTGGCCGCGCAGCAACAGATGGAAGGCCTCCT
GGCGCCGCACCGGCCCAAGGAGCCCGCGTGGTTCCTGGCCACCGT
CGGCGTCTCGCCCGACCACCAGGGCAAGGGTCTGGGCAGCGCCGT
CGTGCTCCCCGGAGTGGAGGCGGCCGAGCGCGCCGGGGTGCCCGC
CTTCCTGGAGACCTCCGCGCCCCGCAACCTCCCCTTCTACGAGCG
GCTCGGCTTCACCGTCACCGCCGACGTCGAGGTGCCCGAAGGACC
GCGCACCTGGTGCATGACCCGCAAGCCCGGTGCCTGATAAGATAC
ATTGATGAGTTTGGACAAACCACAACTAGAATGCAGTGAAAAAAA
TGCTTTATTTGTGAAATTTGTGATGCTATTGCTTTATTTGTAACC
ATTATAAGCTGCAATAAACAAGTTAACAACAACAATTGCATTCAT
TTTATGCTTCAGGTTCAGGGGGAGGTGTGGGAGGTTTTTTAAAGC
AAGTAAAACCTCTACAAATGTGGTATGGCTGATTATGATCCGGCT
GCCTCGCGCGGTACGACGCGTATACCTAGGATCCGGCCGGCCTGC
AGGTGTCCTCACAGGAACGAAGTCCCTAAAGAAACAGTGGCAGCC
AGGTTTAGCCCCGGAATTGACTGGATTCCTTTTTTAGGGCCCATT
GGTATGGCTTTTTCCCCGTATCCCCCCAGGTGTCTGCAGGCTCAA
AGAGCAGCGAGAAGCGTTCAGAGGAAAGCGATCCCGTGCCACCTT
CCCCGTGCCCGGGCTGTCCCCGCACGCTGCCGGCTCGGGGATGCG
GGGGGAGCGCCGGACCGGAGCGGAGCCCCGGGCGGCTCGCTGCTG
CCCCCTAGCGGGGGAGGGACGTAATTACATCCCTGGGGGCTTTGG
GGGGGGGCTGTCCCTCTAGAGCGGCCGCCACCGCGGTGGAGCTCC
AGCTTTTGTTCCCTTTAGTGAGGGTTAATTAGATCTTAATACGAC
TCACTATAGGGCGAATTGGGTACCGGGCCCCCCCTCGAGGTCGAC
GGTATCGATAAGCTTGATATCTATAACAAGAAAATATATATATAA
TAAGTTATCACGTAAGTAGAACATGAAATAACAATATAATTATCG
TATGAGTTAAATCTTAAAAGTCACGTAAAAGATAATCATGCGTCA
TTTTGACTCACGCGGTCGTTATAGTTCAAAATCAGTGACACTTAC
CGCATTGACAAGCACGCCTCACGGGAGCTCCAAGCGGCGACTGAG
ATGTCCTAAATGCACAGCGACGGATTCGCGCTATTTAGAAAGAGA
GAGCAATATTTCAAGAATGCATGCGTCAATTTTACGCAGACTATC
TTTCTAGGGTTAATCTAGCTGCATCAGGATCATATCGTCGGGTCT
TTTTTCCGGCTCAGTCATCGCCCAAGCTGGCGCTATCTGGGCATC
GGGGAGGAAGAAGCCCGTGCCTTTTCCCGCGAGGTTGAAGCGGCA
TGGAAAGAGTTTGCCGAGGATGACTGCTGCTGCATTGACGTTGAG
CGAAAACGCACGTTTACCATGATGATTCGGGAAGGTGTGGCCATG
CACGCCTTTAACGGTGAACTGTTCGTTCAGGCCACCTGGGATACC
AGTTCGTCGCGGCTTTTCCGGACACAGTTCCGGATGGTCAGCCCG
AAGCGCATCAGCAACCCGAACAATACCGGCGACAGCCGGAACTGC
CGTGCCGGTGTGCAGATTAATGACAGCGGTGCGGCGCTGGGATAT
TACGTCAGCGAGGACGGGTATCCTGGCTGGATGCCGCAGAAATGG
ACATGGATACCCCGTGAGTTACCCGGCGGGCGCGCTTGGCGTAAT
CATGGTCATAGCTGTTTCCTGTGTGAAATTGTTATCCGCTCACAA
TTCCACACAACATACGAGCCGGAAGCATAAAGTGTAAAGCCTGGG
GTGCCTAATGAGTGAGCTAACTCACATTAATTGCGTTGCGCTCAC
TGCCCGCTTTCCAGTCGGGAAACCTGTCGTGCCAGCTGCATTAAT
GAATCGGCCAACGCGCGGGGAGAGGCGGTTTGCGTATTGGGCGCT
CTTCCGCTTCCTCGCTCACTGACTCGCTGCGCTCGGTCGTTCGGC
TGCGGCGAGCGGTATCAGCTCACTCAAAGGCGGTAATACGGTTAT
CCACAGAATCAGGGGATAACGCAGGAAAGAACATGTGAGCAAAAG
GCCAGCAAAAGGCCAGGAACCGTAAAAAGGCCGCGTTGCTGGCGT
TTTTCCATAGGCTCCGCCCCCCTGACGAGCATCACAAAAATCGAC
GCTCAAGTCAGAGGTGGCGAAACCCGACAGGACTATAAAGATACC
AGGCGTTTCCCCCTGGAAGCTCCCTCGTGCGCTCTCCTGTTCCGA
CCCTGCCGCTTACCGGATACCTGTCCGCCTTTCTCCCTTCGGGAA
GCGTGGCGCTTTCTCATAGCTCACGCTGTAGGTATCTCAGTTCGG
TGTAGGTCGTTCGCTCCAAGCTGGGCTGTGTGCACGAACCCCCCG
TTCAGCCCGACCGCTGCGCCTTATCCGGTAACTATCGTCTTGAGT
CCAACCCGGTAAGACACGACTTATCGCCACTGGCAGCAGCCACTG
GTAACAGGATTAGCAGAGCGAGGTATGTAGGCGGTGCTACAGAGT
TCTTGAAGTGGTGGCCTAACTACGGCTACACTAGAAGGACAGTAT
TTGGTATCTGCGCTCTGCTGAAGCCAGTTACCTTCGGAAAAAGAG
TTGGTAGCTCTTGATCCGGCAAACAAACCACCGCTGGTAGCGGTG
GTTTTTTTGTTTGCAAGCAGCAGATTACGCGCAGAAAAAAAGGAT
CTCAAGAAGATCCTTTGATCTTTTCTACGGGGTCTGACGCTCAGT
GGAACGAAAACTCACGTTAAGGGATTTTGGTCATGAGATTATCAA
AAAGGATCTTCACCTAGATCCTTTTAAATTAAAAATGAAGTTTTA
AATCAATCTAAAGTATATATGAGTAAACTTGGTCTGACAGTTACC
AATGCTTAATCAGTGAGGCACCTATCTCAGCGATCTGTCTATTTC
GTTCATCCATAGTTGCCTGACTCCCCGTCGTGTAGATAACTACGA
TACGGGAGGGCTTACCATCTGGCCCCAGTGCTGCAATGATACCGC
GAGACCCACGCTCACCGGCTCCAGATTTATCAGCAATAAACCAGC
CAGCCGGAAGGGCCGAGCGCAGAAGTGGTCCTGCAACTTTATCCG
CCTCCATCCAGTCTATTAATTGTTGCCGGGAAGCTAGAGTAAGTA
GTTCGCCAGTTAATAGTTTGCGCAACGTTGTTGCCATTGCTACAG
GCATCGTGGTGTCACGCTCGTCGTTTGGTATGGCTTCATTCAGCT
CCGGTTCCCAACGATCAAGGCGAGTTACATGATCCCCCATGTTGT
GCAAAAAAGCGGTTAGCTCCTTCGGTCCTCCGATCGTTGTCAGAA
GTAAGTTGGCCGCAGTGTTATCACTCATGGTTATGGCAGCACTGC
ATAATTCTCTTACTGTCATGCCATCCGTAAGATGCTTTTCTGTGA
CTGGTGAGTACTCAACCAAGTCATTCTGAGAATAGTGTATGCGGC
GACCGAGTTGCTCTTGCCCGGCGTCAATACGGGATAATACCGCGC
CACATAGCAGAACTTTAAAAGTGCTCATCATTGGAAAACGTTCTT
CGGGGCGAAAACTCTCAAGGATCTTACCGCTGTTGAGATCCAGTT
CGATGTAACCCACTCGTGCACCCAACTGATCTTCAGCATCTTTTA
CTTTCACCAGCGTTTCTGGGTGAGCAAAAACAGGAAGGCAAAATG
CCGCAAAAAAGGGAATAAGGGCGACACGGAAATGTTGAATACTCA
T

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

1. A method for increasing stem cell differentiation to produce erythromyeloid progenitors, megakaryocytes and NK cells but not differentiated T cells, comprising enforcing expression of SOX18 in hemogenic endothelial cells during their transition from endothelium to hematopoietic cell progenitors.

2. The method of claim 1, wherein the cells produced are enriched in NK progenitor cells.

3. The method of claim 2, wherein NK progenitor cell production is increased about five-fold compared with hemogenic epithelial cells wherein SOX18 production is not enforced.

4. The method of claim 1, wherein SOX18 expression is enforced after introduction of an inducible SOX18-encoding expression construct.

5. The method of claim 1, wherein SOX18 expression is enforced after introduction of a constitutively expressing SOX18-encoding expression construct.

6. The method of claim 1, wherein SOX18 expression is enforced by introducing a modified mRNA (mmRNA) encoding SOX18 into the cell.

7. The method of claim 4, wherein the inducible SOX18 expression construct is enforced by contacting the cells with doxycycline.

8. A NK progenitor cell culture produced by the method of claim 1.

9. A composition comprising the NK progenitor cell culture of claim 8.

10. A pharmaceutical composition comprising differentiated NK cells produced from the composition of claim 9 and pharmaceutically (or therapeutically) acceptable excipients and adjuvants.

11. An immunotherapeutic method comprising administration of a therapeutically effective amount of the pharmaceutical composition of claim 10 to an individual in need thereof.

12. A megakaryocyte progenitor cell culture produced by the method of claim 1.

13. A composition comprising the megakaryocyte progenitor cell culture of claim 12.

14. A pharmaceutical composition comprising differentiated megakaryocyte cells produced from the composition of claim 13 and pharmaceutically (or therapeutically) acceptable excipients and adjuvants.

15. An immunotherapeutic method comprising administration of a therapeutically effective amount of the pharmaceutical composition of claim 14 to an individual in need thereof.

16. A composition comprising a differentiated NK cell population produced according to the method of claim 1.

17. A composition comprising a differentiated megakaryocyte cell population produced according to the method of claim 1.


SEQUENCE LISTING (20 sequences)

SEQ ID NO: 1 - DNA, 22 bp, synthetic construct
  source 1..22: mol_type = other DNA
ccgaacagggacttgtttagag

SEQ ID NO: 2 - DNA, 22 bp, synthetic construct
  source 1..22: mol_type = other DNA
aggttggctctggcatttatag

SEQ ID NO: 3 - DNA, 20 bp, synthetic construct
  source 1..20: mol_type = other DNA
cagtgggcagcgaagctaca

SEQ ID NO: 4 - DNA, 22 bp, synthetic construct
  source 1..22: mol_type = other DNA
acaggcagtggtagccatcctc

SEQ ID NO: 5 - DNA, 22 bp, synthetic construct
  source 1..22: mol_type = other DNA
ggactatcggagtttgggattt

SEQ ID NO: 6 - DNA, 20 bp, synthetic construct
  source 1..20: mol_type = other DNA
tgggaagcgtagttgttgag

SEQ ID NO: 7 - DNA, 20 bp, synthetic construct
  source 1..20: mol_type = other DNA
aggtccaggatcagggtatt

SEQ ID NO: 8 - DNA, 22 bp, synthetic construct
  source 1..22: mol_type = other DNA
ccagagaaggagggattgattc

SEQ ID NO: 9 - DNA, 20 bp, synthetic construct
  source 1..20: mol_type = other DNA
gcgccttctctgaaaacaat

SEQ ID NO: 10 - DNA, 20 bp, synthetic construct
  source 1..20: mol_type = other DNA
cagttccagggtctggtgtt

SEQ ID NO: 11 - DNA, 20 bp, synthetic construct
  source 1..20: mol_type = other DNA
gcaaagagtggtcggaagaa

SEQ ID NO: 12 - DNA, 24 bp, synthetic construct
  source 1..24: mol_type = other DNA
cgctctcgagtaaggtacatattg

SEQ ID NO: 13 - DNA, 23 bp, synthetic construct
  source 1..23: mol_type = other DNA
cctggaggagaagaggaaagaga

SEQ ID NO: 14 - DNA, 25 bp, synthetic construct
  source 1..25: mol_type = other DNA
ttgaggacctctgtgtatttgtcaa

SEQ ID NO: 15 - DNA, 9190 bp, synthetic construct
  source 1..9190: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaattcctgcagcccgggggatccactagttctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccaaggccagcttcccacaataagttgggtgaattttggctcattcctcctttctataggattgaggtcagagctttgtgatgggaattctgtggaatgtgtgtcagttagggtgtggaaagtcccgcgatcgatcctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccatctagagcaactagcatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaatctaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcatgcatcatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcccatgattacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgggtccagggttctcctccacgtctccagcctgcttcagcaggctgaagttagtagctccgcttccgtatacgccggagatgcacgcgctgtaatagaccgcgctgctggcgtccgacagcgcggagatcaggctgctctcctctgggcaggacatggcgcgcgggcccagtttggccagtgccacgtggtacgggagcccgggggcgtcgggccgagtccggctgcagttgaggtactggtcgaactcggtgaggtccacgtcggcccacagatcggcggcgggccccagcggctcggcgctctccagcggcggggcctcgggcggcggcgacagcgggccggggtacgggccgggcgtgcccagggtgccgtagtacaggccagcgagcggcgccgcggggggcgcggtcctgagcgcctccgccaggggagccccgtagcaaccgccggggtcccgcgacaactcggtgggcgcgtagggcgcgcggaagggccgcagcgcgcagtcctcgggcgccgcgggcggtgggaagaaggcagcctcgccgggctccaggccgtccagaggcgagcgctcgggcgtgggcagccccaggccgtcgaactcggcgcccagcgggggcagctcgcggaaggcgcgagccgagccagacgccgcggggaaaggctcgggcggtggctgcgggggcgctaatcccgggagcaggaggccgggctccagccgccgggccttgcgcgcctgcttcttgcggcgcggccggtacttgtagttggggtggtcgcgcaagtgctgcacgcgcagccgttcggcttcctccacgaagggccgcttctccgccgcgttcagctccttccacgctttgcccagcatcttgctgagcaccgcgttgtgcaggtccgggttctgctgagccagccgcttgcgctcgtcctttgcccacaccatgaaggcgttcatgggccgccggatgcgcgactcgtctgccgcctggcgttccccgcggccggccgggctgaggccatagcgccccggctcggggctgcgcgggggactgcgctgcgggctgggcggcgaggcgggcgcggcgggcgcggcgagggcggcggggccggcggcgaggccgcgcgtgtcagcggcggccccgtgtcccggggcccatgcacagtcgcggcgggcgggcgggtcgtcctgtgcgccgtagccgggcggcgatctctgcatggatcctccaggcgatctgacggttcactaaacgagctctgcttatataggcctcccaccgtacacgcctacctcgacatacgttctctatcactgatagggagtaaactcgacatacgttctctatcactgatagggataaactcgacatacgttctctatcactgatagggagtaaactcgacatacgttctctatcactgatagggagtaaactcgacatacgttctctatcactgatagggagtaaactcgacatcgttctctatcactgatagggagtaaactcgacatacgttctctatcactgatagggagtaaactcgctcgagacgcgtatacctaggatccggccggcctgcaggtgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctagagcggccgccaccgcggtggagctccagcttttgttccctttagtgagggttaattagatcttaatacgactcactatagggcgaattgggtaccgggccccccctcgaggtcgacggtatcgataagcttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 16 - DNA, 1152 bp, synthetic construct
  source 1..1152: mol_type = other DNA
atgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggc

SEQ ID NO: 17 - protein, 384 aa, synthetic construct [sequence omitted]

SEQ ID NO: 18 - DNA, 7741 bp, synthetic construct
  source 1..7741: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaattcctgcagcccgggggatccactagttctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccaaggccagcttcccacaataagttgggtgaattttggctcattcctcctttctataggattgaggtcagagctttgtgatgggaattctgtggaatgtgtgtcagttagggtgtggaaagtcccgcgatcgtcgagagatttctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccatctagagcaactagcttgggcccgggaattaattcaccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggaattcggaagcggagagggcagaggaagtcttctaacatgcggtgacgtggaggagaatcccggccctgatatcatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgcttcaggttcagggggaggtgtgggaggttttttaaagcaagtaaaacctctacaaatgtggtatggctgattatgatccggctgcctcgcgcggtacgacgcgtatacctaggatccggccggcctgcaggtgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctagagcggccgccaccgcggtggagctccagcttttgttccctttagtgagggttaattagatcttaatacgactcactatagggcgaattgggtaccgggccccccctcgaggtcgacggtatcgataagcttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 19 - DNA, 17 bp, synthetic construct
  source 1..17: mol_type = other DNA
ggcaaagcgtggaagga

SEQ ID NO: 20 - DNA, 19 bp, synthetic construct
  source 1..19: mol_type = other DNA
cggccggtacttgtagttg